SEQUENCE LISTING (9 sequences)

SEQ ID NO: 1 - DNA, 20 bp, Artificial
  Synthetic PCR primer
gccttcactgctgctcatga

SEQ ID NO: 2 - DNA, 22 bp, Artificial
  Synthetic PCR primer
ccaacacatggctttgaattgt

SEQ ID NO: 3 - DNA, 25 bp, Artificial
  Synthetic oligonucleotide probe
ctgggccatgtcttcaacatgctcc

SEQ ID NO: 4 - DNA, 22 bp, Artificial
  Synthetic PCR primer
aagctgcttatgaggtttccca

SEQ ID NO: 5 - DNA, 22 bp, Artificial
  Synthetic PCR primer
gggtatccgtagagcacatcct

SEQ ID NO: 6 - DNA, 33 bp, Artificial
  SYnthetic oligonucleotide probe
agcccagtacttattacctttgaaaaaccggac

SEQ ID NO: 7 - DNA, 21 bp, Artificial
  Synthetic PCR primer
aacactggacgaaggatgcat

SEQ ID NO: 8 - DNA, 21 bp, Artificial
  Synthetic PCR primer
acccagggaatgaccaagttc

SEQ ID NO: 9 - DNA, 30 bp, Artificial
  Synthetic oligonucleotide probe
agggatcaattttctccttgttgctgctca

(19) United States
(12) Patent Application Publication
(10) Pub. No.: US 2009/0104155 A1
(43) Pub. Date: Apr. 23, 2009

(54) TREATMENT OF CONNECTIVE TISSUE DISORDERS

(76) Inventors: Laurie R. Goodrich, Fort Collins, CO (US); Richard Jude Samulski, Chapel Hill, NC (US); Vivian W. Choi, Carrboro, NC (US)

Correspondence Address: MYERS BIGEL SIBLEY & SAJOVEC, PO BOX 37428, RALEIGH, NC, 27627, US

(21) Appl. No.: 12/297,668

(22) PCT Filed: Apr. 20, 2007
(86) PCT No.: PCT/US2007/009821

§ 371 Date: Dec. 18, 2008

Related U.S. Application Data
(60) Provisional application No. 60/794,046, filed on Apr. 21, 2006.

(51) Int. Cl. A61K 48/00; C12N 15/86; A61P 19/04

(52) U.S. Cl. 424/93.2; 435/456

(57) ABSTRACT

The present invention relates to methods of delivering nucleic acids to connective tissue cells and to methods of treating connective tissue disorders; in particular, the invention provides methods of delivering nucleic acids to connective tissue cells and methods of treating connective tissue disorders using parvovirus vectors.

### STATEMENT OF PRIORITY

The present application claims the benefit, under 35 U.S.C. § 119(e), of U.S. Provisional Application Ser. No. 60/794,046, filed Apr. 21, 2006, the entire contents of which are incorporated by reference herein.

### FIELD OF THE INVENTION

The present invention relates to methods of delivering nucleic acids to connective tissue cells and to methods of treating connective tissue disorders; in particular, the invention provides methods of delivering nucleic acids to connective tissue cells and methods of treating connective tissue disorders using parvovirus vectors.

### BACKGROUND OF THE INVENTION

It is becoming clear that vectors based upon adeno-associated virus (AAV) are the vectors of choice for certain gene therapy applications. The utilization of AAV vectors in such protocols is based on the advantageous properties of AAV, including lack of pathogenicity and pathology, ease of preparation and purification, long term expression in many tissues, and lack of a detrimental cell-mediated immune response.

AAV serotype 2 (AAV2) is the best studied of the AAV isolates. Over the past decade, inroads have been made in the evaluation of the tissue tropism of alternative AAV serotypes. These studies have shown that distinct AAV serotypes may be better suited for particular applications. For example, serotypes 1, 6 and 7 are the most promising for delivery to skeletal muscle. To illustrate, as compared with AAV2, AAV1 can be administered to skeletal muscle at lower dosages (i.e., fewer particles) and can express the transgene at earlier time points and at higher levels of expression.

The extensive development of AAV2 as a vector has been facilitated by 30 years of studying its biology in vitro. Recombinant AAV2 (rAAV2) has proven to be a suitable gene transfer vector in many different organisms (Monohan and Samulski (2000) Gene Ther. 7:24, Rabinowitz and Samulski (1998) Curr. Opin. Biotechnol. 9:470). As the number of applications evaluating gene transfer increases in vitro and in vivo, limitations to efficient rAAV2 transduction have become apparent (Bartlett et al. (2000) J. Virol. 74:2777, Davidson et al. (2000) Proc. Natl. Acad. Sci. USA 97:3428, Hansen et al. (2001) J. Virol. 75:4080, Samulski et al. (1999) in, Adeno-associated viral vectors Cold Spring Harbor, N.Y., Walters et al. (2000) J. Virol 74:535, Xiao et al. (1999) J. Virol. 73:3994, Zabner et al. (2000) J. Virol., 74:3852). The natural tropism of any virus, including rAAV2, is a fundamental limitation to efficient gene transfer.

With the identification of the AAV2 receptor, the requirements for efficient entry in target cells have become a critical topic of study (Summerford and Samulski (1998) J. Virol. 72:1438). Efforts have been made to overcome these restrictions by broadening the host range using either bispecific antibodies to the virion shell (Bartlett et al. (1999) Nat. Biotechnol. 17:181) or through capsid insertional mutagenesis (International patent publication WO 00/28004; Rabinowitz et al. (1999) Virology 265:274; Girod et al. (1999) Nat. Med. 5:1052, Wu et al. (2000) J. Virol. 74:8635). While these efforts are beginning to bear fruit, utilizing the other serotypes of AAV may yet provide additional resources for making safe and efficient gene transfer vectors.

Duplexed parvovirus vectors are described in international patent publication WO 01/92551 and McCarty et al., (2003) Gene Therapy 10:2112-2118. In at least some tissues, duplexed parvovirus vectors display a more rapid onset and/or a higher level of transgene expression than do conventional single-stranded rAAV vectors, presumably because the duplexed vector circumvents the rate-limiting step of second-strand synthesis in these cells.

### SUMMARY OF THE INVENTION

As one aspect, the invention provides a method of delivering a nucleic acid to a connective tissue cell, the method comprising contacting the cell with a virus vector comprising:
(a) an adeno-associated virus (AAV) capsid; and
(b) a recombinant nucleic acid comprising 5′ and 3′ AAV terminal repeats and a heterologous nucleotide sequence; wherein the recombinant nucleic acid is packaged within the AAV capsid.

In particular embodiments, the cell is contacted in vitro. Accordingly, as another aspect, the invention provides a method of delivering a nucleic acid to a connective tissue of a subject, the method comprising administering to the subject a cell contacted in vitro with a virus vector according to the foregoing method.

The invention also provides a method of treating a connective tissue disorder in a subject, the method comprising administering to a subject in need thereof an effective amount of a cell contacted in vitro with a virus vector according to the foregoing method.

As a further aspect, the invention provides a method of administering a nucleic acid to a connective tissue in a subject, the method comprising administering to the subject a virus vector comprising:
(a) an AAV capsid; and
(b) a recombinant nucleic acid comprising 5′ and 3′ AAV terminal repeats and a heterologous nucleotide sequence; wherein the recombinant nucleic acid is packaged within the AAV capsid.

As yet another aspect, the invention provides a method of treating a connective tissue disorder, the method comprising administering to a subject in need thereof an effective amount of a virus vector comprising:
(a) an AAV capsid; and
(b) a recombinant nucleic acid comprising 5′ and 3′ AAV terminal repeats and a heterologous nucleotide sequence; wherein the recombinant nucleic acid is packaged within the AAV capsid.

The present invention further provides for the use of a virus vector or a cell of the invention in the manufacture of a medicament for the treatment of a connective tissue disorder (e.g., a joint disorder), to enhance cartilage healing and/or regeneration and/or to reduce joint inflammation. Optionally, the virus vector is a duplexed parvovirus vector.

These and other aspects of the invention are set forth in more detail in the following description of the invention.

### BRIEF DESCRIPTION OF THE DRAWINGS

FIG. 1 shows a fluorescence photomicrograph of chondrocytes at day 7 following transduction with AAV-GFP serotypes 1, 2, 3, 4, 5, 6, and 8 (columns going from the left to right: serotype 1 (S1), 2 (S2), 3 (S3), 4 (S4), 5 (S5), 6 (S6) and 8 (S8)). The top row represents 10,000 viral particles per cell, the second row 1000, third row 100, and bottom row 10 viral particles per cell.

FIG. 2 shows a fluorescence photomicrograph of synoviocytes at day 7 following transduction with AAV-GFP serotypes 1, 2, 3, 4, 5, 6 and 8 (columns going from the left to right: serotype 1 (S1), S2, S3, S4, S5, S6 and S8). The top row represents 10,000 viral particles per cell, the second row 1000, third row 100, and bottom row 10 viral particles per cell.

FIG. 3 shows a fluorescence photomicrograph representing the second set of chondrocyte transductions for AAV-GFP serotypes 2, 3, 5 and 6 (columns going from the left to right: serotype 2 (S2), S3, S5 and S6). The top row represents 8000 viral particles per cell, second row 4000, third row 2000, fourth row 1000, and bottom row 500.

FIG. 4 shows a fluorescence photomicrograph representing the second set of synoviocyte transductions for AAV-GFP serotypes 2, 3, 5 and 6 (columns going from the left to right: serotype 2 (S2), S3, S5 and S6). The top row represents 8000 viral particles per cell, second row 4000, third row 2000, fourth row 1000, and bottom row 500.

FIG. 5 represents transduction efficiencies for AAV serotype 2 (S2), S3, S5 and S6 in chondrocytes (top graph) and synoviocytes (bottom graph) up to passage 3 (day 51).

FIG. 6 represents the relative fluorescence of chondrocytes (top graph) and synoviocytes (bottom graph) measured with the fluorometer from day 1 through day 7 for AAV-GFP serotype 2 (S2), S3, S5 and S6 at 4000 viral particles per cell for all serotypes.

FIG. 7 represents cell viability for chondrocytes (top graph) and synoviocytes (bottom graph). Cell viability was determined at passages 1 (day 15), 2 (day 36) and 3 (day 52).

FIG. 8 shows RNA expression profiles for inflammatory molecules in chondrocytes transduced with AAV serotype 6 (S6).

FIG. 9 shows RNA expression profiles for inflammatory molecules in synoviocytes transduced with AAV serotype 3 (S3).

FIG. 10 shows RNA expression profiles for inflammatory molecules in chondrocytes transduced with AAV serotype 2 (S2).

FIG. 11A shows the expression of IGF under various growth conditions in rAAV-IGF-I S6-transduced chondrocytes from equine stifle joints in alginate culture. FIG. 11B shows the expression of IGF under various growth conditions in rAAV-IGF-I S3-transduced synoviocytes from equine stifle joints in alginate culture.

FIG. 12A shows cell growth of rAAV-IGF-I S6-transduced chondrocytes in alginate at 0, 3, 7, 10 and 14 days. FIG. 12B shows cell growth of rAAV-IGF-I S3-transduced synoviocytes in alginate at 0, 3, 7, 10 and 14 days.

FIG. 13A shows the determination of optimal alginate concentration for cell growth and IGF production of rAAV-IGF-I S6-transduced chondrocytes. FIG. 13B shows the determination of optimal alginate concentration for cell growth and IGF production of rAAV-IGF-I S3-transduced synoviocytes.

FIGS. 14A and 14B show the transduction efficiency in chondrocytes of AAV-GFP S2, S3, S5 and S6 at various titers on days 7 and 14, respectively. FIGS. 14C and 14D show the transduction efficiency in synoviocytes of AAV-GFP S2, S3, S5 and S6 at various titers on days 7 and 14, respectively.

FIGS. 15A-D shows RNA expression profiles for inflammatory molecules in chondrocytes transduced with various AAV-GFP serotypes.

FIGS. 16A-D shows RNA expression profiles for inflammatory molecules in synoviocytes transduced with various AAV-GFP serotypes.

FIGS. 17A-B show the viability (A) and transduction efficiency (B) of mesenchymal stem cells (MSCs) with AAV serotypes S1-8.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety.

Except as otherwise indicated, standard methods known to those skilled in the art may be used for the construction of rAAV constructs, packaging vectors expressing the AAV rep and/or cap sequences, and transiently and stably transfected packaging cells, and production of recombinant viral vectors. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

### DEFINITIONS

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Furthermore, the term “about,” as used herein when referring to a measurable value such as an amount of a compound or agent of this invention, dose, time, temperature, and the like, is meant to encompass variations of ±20%, ±10%, ±5%, ±1%, ±0.5%, or even ±0.1% of the specified amount.

The term “parvovirus” as used herein encompasses the family Parvoviridae, including autonomously-replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV) includes but is not limited to, AAV serotype 1 (AAV1), AAV2, AAV3 (including types 3A and 3B), AAV4, AAV5, AAV6, AAV7, AAV8, AAV9, AAV10, AAV11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). Recently, a number of putative new AAV serotypes and clades have been identified (see, e.g., Gao et al., (2004) J. Virology 78:6381-6388; Moris et al., (2004) Virology 33-:375-383; and Table 1).

The genomic sequences of the various serotypes of AAV and the autonomous parvoviruses, as well as the sequences of the terminal repeats, Rep proteins, and capsid subunits are known in the art. Such sequences may be found in the literature or in public databases such as GenBank. See, e.g., GenBank Accession Numbers NC_{—}002077, NC_{—}001401, NC_{—}001729, NC_{—}001863, NC_{—}001829, NC_{—}001862, NC_{—}000883, NC_{—}001701, NC_{—}001510, NC_{—}006152, NC_{—}006261, AF063497, U89790, AF043303, AF028705, AF028704, J02275, J01901, J02275, X01457, AF288061, AH009962, AY028226, AY028223, NC_{—}001358, NC_{—}001540, AF513851, AF513852, AY530579; the disclosures of which are incorporated by reference herein for teaching parvovirus and AAV nucleic acid and amino acid sequences. See also, e.g., Srivistava et al., (1983) J. Virology 45:555; Chiorini et al., (1998) J. Virology 71:6823; Chiorini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1. As used herein, the term “polypeptide” encompasses both peptides and proteins, unless indicated otherwise.

A “nucleic acid” or “nucleic acid sequence” or “nucleotide sequence” (or similar terms) is a sequence of nucleotide bases, and may be RNA, DNA or DNA-RNA hybrid sequences (including both naturally occurring and non-naturally occurring nucleotide), but are preferably either single or double stranded DNA sequences.

As used herein, an “isolated” nucleotide sequence or nucleic acid or similar term (e.g., an “isolated DNA” or an “isolated RNA”) means a nucleotide sequence or nucleic acid that is at least partially separated from at least some of the other components of the naturally occurring organism or virus, for example, the cell or viral structural components or other polypeptides or nucleic acids commonly found associated with the nucleic acid.

A “heterologous nucleotide sequence” or “heterologous nucleic acid” (and similar terms) is a sequence that is not naturally occurring in the virus and/or the cell into which it is introduced to be expressed. Generally, the heterologous nucleotide sequence or nucleic acid comprises an open reading frame that encodes a polypeptide or nontranslated RNA of interest (e.g., for delivery to a cell or subject).

An “isolated polypeptide” means a polypeptide that is at least partially separated from at least some of the other components of the naturally occurring organism or virus, for example, the cell or viral structural components or other polypeptides or nucleic acids commonly found associated with the polypeptide.

A “therapeutic polypeptide” is a polypeptide that can alleviate, reduce, delay and/or prevent symptoms that result from an absence or defect in a protein in a cell or subject. Alternatively, a “therapeutic polypeptide” is one that otherwise confers a benefit to a subject, e.g., anti-cancer effects or improvement in transplant survivability.

As used herein, an “isolated cell” is a cell that has been removed from a subject or is derived from a cell that has been removed from a subject, and has been enriched or at least partially purified from the tissue or organ (e.g., cartilage, synovium, meniscus, bone marrow) with which it is associated in its native state.

As used herein, an “effective amount” refers to an amount of a virus vector, cell or pharmaceutical composition that is sufficient to produce a desired effect, which is optionally a therapeutic and/or prophylactic effect (i.e., by administration of a treatment effective amount). For example, an “effective amount” can be an amount that is sufficient to treat a connective tissue disorder.

A “treatment effective” amount as used herein is an amount that is sufficient to provide some improvement or benefit to the subject. Alternatively stated, a “treatment effective” amount is an amount that will provide some alleviation, mitigation, or decrease in at least one clinical symptom in the subject. Those skilled in the art will appreciate that the therapeutic effects need not be complete or curative, as long as some benefit is provided to the subject.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition and/or prevention or delay of the onset of a disease or disorder. Thus, the terms “treat,”, “treating” or “treatment of” (or grammatical variations thereof) refer to both prophylactic and therapeutic regimens.

As used herein, the terms “virus vector,” “vector” or “gene delivery vector” (or similar terms) refer to a virus (e.g., AAV) particle that functions as a nucleic acid delivery vehicle, and which comprises the vector genome (e.g., viral DNA [vDNA]) packaged within an AAV capsid. Alternatively, in some contexts, the term “vector” may be used to refer to the vector genome/vDNA alone.

A “rAAV vector genome” or “rAAV genome” is a recombinant AAV genome (i.e., vDNA) that comprises one or more heterologous nucleotide sequences. Typically, the rAAV vector genome only retains the minimal terminal repeat sequence(s) (each 145 bases) so as to maximize the size of the transgene that can be efficiently packaged by the vector. All other viral structural and non-structural coding sequences are dispensable and may be supplied in trans (Muzyczka, (1992) Curr. Topics Microbiol. Immunol 158:97), e.g., from a vector, such as a plasmid, or by stably integrating the sequences into a packaging cell. The rAAV vector genome generally comprises at least one AAV terminal repeat sequence, optionally two AAV terminal repeat sequences, which typically will be at the 5′ and 3′ ends of the heterologous nucleotide sequence(s), but need not be contiguous thereto. The terminal repeats can be the same or different from each other.

The term “terminal repeat” includes any viral terminal repeat and/or partially or completely synthetic sequences that form hairpin structures and function as an inverted terminal repeat, such as the “double-D sequence” as described in U.S. Pat. No. 5,478,745 to Samulski et al.

Ari “AAV terminal repeat” may be from any AAV, including but not limited to serotypes 1, 2, 3, 4, 5, 6, 7, 8, 9, 10, 11 or 12 or any other AAV now known or later discovered. The AAV terminal repeat need not have a wild-type sequence (e.g., a wild-type sequence may be altered by insertion, deletion, truncation or missense mutations), as long as the terminal repeat mediates the desired functions, e.g., replication, nicking, virus packaging, integration, and/or provirus rescue, and the like.

A “non-resolvable terminal repeat” encompasses naturally-occurring terminal repeat sequences (including altered forms thereof) and, for example, can be derived from a parvovirus, including an AAV, or can be from another virus or, as a further alternative, can be partially or completely synthetic. By “non-resolvable terminal repeat” it is meant that the terminal repeat is not recognized by and resolved (i.e., “nicked”) by the AAV Rep proteins, such that resolution of the terminal repeat is substantially reduced (e.g., by at least about 50%, 60%, 70%, 80%, 90%, 95%, 98% or greater as compared with a resolvable terminal repeat) or eliminated.

The non-resolvable terminal repeat may be a non-AAV viral sequence that is not recognized by the AAV Rep proteins, or it can be an AAV terminal repeat that has been modified (e.g., by insertion, substitution and/or deletion) so that it is no longer recognized by the AAV Rep proteins. Further, a non-resolvable terminal repeat can be any terminal repeat that is non-resolvable under the conditions used to produce the virus vector. For example, the non-resolvable terminal repeat may not be recognized by the Rep proteins used to replicate the vector genome. To illustrate, the non-resolvable terminal repeat can be an autonomous parvovirus terminal repeat or a virus terminal repeat other than a parvovirus terminal repeat that is not recognized by AAV Rep proteins. As another illustrative example, the resolvable terminal repeat and Rep proteins may be from one AAV serotype (e.g., AAV2), and the non-resolvable terminal repeat is from another AAV serotype (e.g., AAV5) that is not recognized by the AAV2 Rep proteins, such that resolution is substantially reduced or eliminated. Further, an AAV terminal repeat can be modified so that resolution by the AAV Rep proteins is substantially reduced or eliminated.

As a yet further alternative, the non-resolvable terminal repeat can be any inverted repeat sequence that forms a hairpin structure and cannot be nicked by the AAV Rep proteins.

### Virus Vectors for Delivery to Connective Tissue.

The present invention provides virus vectors, cells, pharmaceutical formulations, and methods for delivering a nucleic acid of interest to a connective tissue cell in vivo or in vitro (the latter including ex vivo).

As described herein, the rAAV vector can be any suitable rAAV vector now known or later discovered. In general, the rAAV vector comprises an AAV capsid, which packages a recombinant vector genome that comprises at least one AAV terminal repeat (optionally two AAV terminal repeats) and a heterologous nucleotide sequence of interest.

The virus vector of the invention can be a “targeted” parvovirus vector (i.e., the AAV capsid comprises an exogenous targeting sequence), a “chimeric” parvovirus vector (i.e., the AAV capsid comprises a capsid region from a different AAV or autonomous parvovirus) and/or a “hybrid” parvovirus vector (i.e., in which the AAV capsid and the AAV terminal repeat(s) are from different AAV) as described in international patent publication WO 00/28004 and Chao et al., (2000) Molecular Therapy 2:619.

The virus vector of the invention can further be a duplexed parvovirus vector as described in international patent publication WO 01/92551 and McCarty et al., (2003) Gene Therapy 10:2112-2118.

In addition, the AAV capsid or vector genome can contain other modifications, including insertions, deletions and/or substitutions.

In particular embodiments, the rAAV vector comprises an AAV capsid as listed in Table 1 including but limited to an AAV1, AAV2, AAV3, AAV4, AAV5, AAV6, AAV7, AAV8, AAV9, AAV10, AAV11 or AAV12 capsid, including modified forms thereof. Optionally, the capsid can be an AAV2, AAV3 or AAV6 capsid or a modified form thereof.

The vector genome can comprise one or more (e.g., two) AAV terminal repeats, which may be the same or different. Further, the one or more AAV terminal repeats can be from the same AAV serotype as the AAV capsid, or can be different. In particular embodiments, the vector genome comprises an AAV1, AAV2, AAV3, AAV4, AAV5, AAV6, AAV7, AAV8, AAV9, AAV10, AAV11 and/or AAV12 terminal repeat.

In representative embodiments of the invention, the virus vector is a duplexed parvovirus vector, wherein the recombinant vector genome comprises 5′ and 3′ AAV terminal repeats (that are resolvable), a heterologous nucleotide sequence of interest, and a non-resolvable terminal repeat. Duplexed parvovirus vectors and their production are described in international patent publication WO 01/92551 and McCarty et al., (2003) Gene Therapy 10:2112-2118.

In general, duplexed parvovirus vectors are dimeric self-complementary (sc) polynucleotides (typically, DNA) packaged within an AAV capsid. In some respects, the recombinant viral genome that is packaged within the capsid is essentially a “trapped” AAV replication intermediate that cannot be resolved to produce the plus and minus polarity strands. Duplexed parvovirus vectors appear to circumvent the need for host cell mediated synthesis of complementary DNA inherent in conventional rAAV vectors, thereby addressing one of the limitations of rAAV vectors.

The duplexed parvovirus vectors are fundamentally different from conventional rAAV vectors, and from the parent AAV, in that the viral DNA may form a double-stranded hairpin structure due to intrastrand base pairing, and the DNA strands of both polarities are encapsidated. Thus, the duplexed parvovirus vector is functionally similar to double-stranded DNA virus vectors rather than the AAV from which it was derived. This feature addresses a previously recognized shortcoming of rAAV mediated gene transfer, which is the limited propensity of the desired target cell to synthesize complementary DNA to the single-stranded genome normally encapsidated by AAV.

While not wishing to be held to any particular theory of the invention, it is possible that the virion genome is retained in a single-stranded form while packaged within the viral capsid. Upon release from the capsid during viral infection, it appears that the dimeric molecule “snaps back” or anneals to form a double-stranded molecule by intra-strand basepairing, with the non-resolvable TR sequence forming a covalently-closed hairpin structure at one end. This double-stranded viral DNA obviates the need for host cell mediated second-strand synthesis, which has been postulated to be a rate-limiting step for AAV transduction.

In the case of connective tissue cells, duplexed parvovirus vectors may be advantageous because they may provide a faster onset of gene expression and/or higher levels of gene expression, thereby permitting lower dosages, which in turn may result in a reduced likelihood and/or extent of inflammation in target tissues.

The duplexed parvovirus vector genome generally comprises in the 5′ to 3′ direction, (i) a resolvable AAV terminal repeat, (ii) a heterologous nucleotide sequence of interest (coding or noncoding strand), (iii) a non-resolvable terminal repeat, (iv) a complementary sequence or substantially complementary (e.g., at least about 90%, 95%, 98%, 99% or more) sequence to the heterologous nucleotide sequence of interest of (ii), and (v) a resolvable AAV terminal repeat. Those skilled in the art will appreciate that the vector genome can comprise other sequences (e.g., intervening sequences between the sequences specifically described above).

In particular embodiments, the sequences in each half of the vector genome (e.g., the entire sequence or the sequences between the AAV terminal repeat and the non-resolvable terminal repeat) are substantially complementary (i.e., at least about 90%, 95%, 98%, 99% nucleotide sequence complementarity or more), so that the vector genome may form double-stranded molecules due to base-pairing between the complementary sequences. In other words, the vector genome is essentially an inverted repeat with the two halves joined by the non-resolvable terminal repeat. In particular embodiments, the two halves of the vector genome (i.e., the entire sequence or the sequences between the AAV terminal repeats and the non-resolvable terminal repeat) are essentially completely self-complementary (i.e., contain an insignificant number of mismatched bases) or completely self-complementary.

In other embodiments, the two strands of the heterologous nucleotide sequence of interest (with or without regulatory elements) are substantially complementary (i.e., at least about 90%, 95%, 98%, 99% nucleotide sequence complementarity or more). In particular embodiments, the two strands of the heterologous nucleotide sequence(s) are essentially completely self-complementary (i.e., contain an insignificant number of mismatched bases) or completely self-complementary.

In general, the vector genome of the duplexed parvoviruses can contain positions or regions of non-complementarity to the extent that expression of the heterologous nucleotide sequence(s) from the duplexed parvovirus vector is enhanced (e.g., earlier onset and/or higher level of expression) than from a corresponding rAAV vector. The duplexed parvoviruses of the present invention provide the host cell with a double-stranded molecule that addresses one of the drawbacks of rAAV vectors, i.e., the need for the host cell to convert the single-stranded rAAV virion DNA into a double-stranded DNA. The presence of any substantial regions of non-complementarity within the virion DNA, in particular, within the heterologous nucleotide sequence(s) may be recognized by the host cell, and may result in DNA repair mechanisms being recruited to correct the mismatched bases, thereby counteracting the advantageous characteristics of the duplexed parvovirus vectors, e.g., reduction or elimination of the need for the host cell to process the viral template.

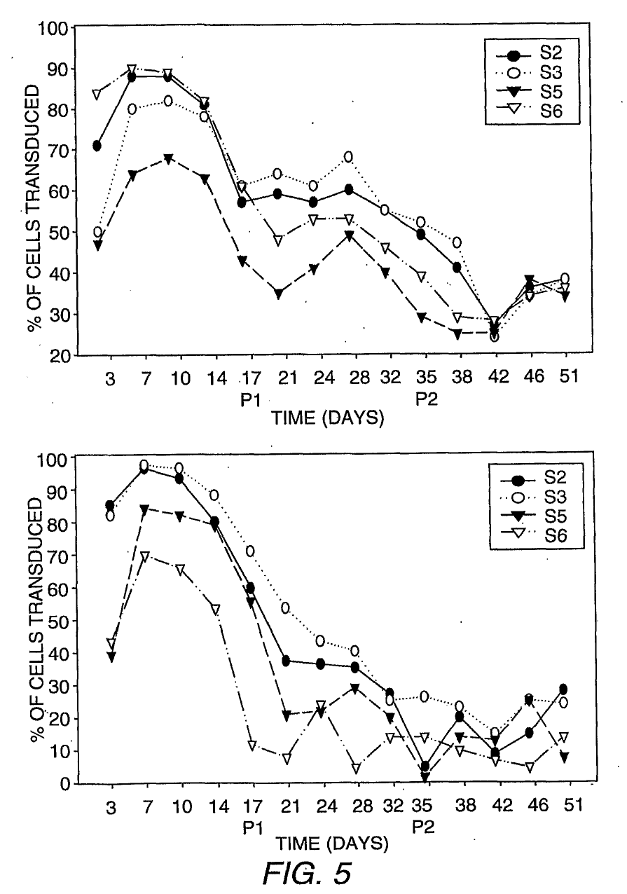

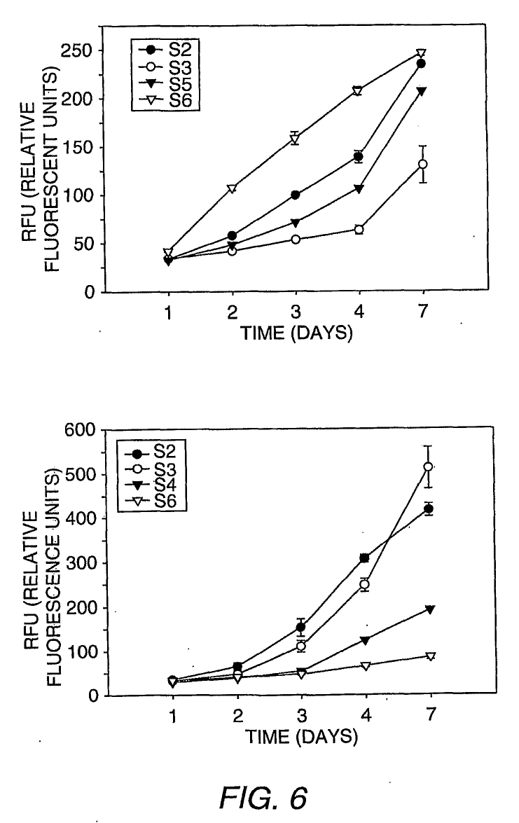

A non-resolvable AAV terminal repeat can be produced by any method known in the art. For example, insertion into the terminal repeat will displace the nicking site (i.e., trs) and result in a non-resolvable terminal repeat. The designation of the various regions or elements within the terminal repeat are known in the art (see, e.g., BERNARD N. FIELDS et al., VIROLOGY, volume 2, chapter 69, FIG. 5, 3d ed., Lippincott-Raven Publishers and FIG. 6 of WO 01/925551). An insertion can also be made into the sequence of the terminal resolution site (trs). Alternatively, an insertion can be made at a site between the Rep Binding Element (RBE) within the A element and the trs (see, FIG. 6 of WO 01/925551). The core sequence of the AAV trs site is known in the art and has been described (Snyder et al., (1990) Cell 60:105; Snyder et al., (1993). J. Virology 67:6096; Brister & Muzyczka, (2000) J. Virology 74:7762; Brister & Muzyczka, (1999) J. Virology 73:9325. For example, Brister & Muzyczka, (1999) J. Virology 73:9325, describes a core trs sequence of 3′-CCGGT/TG-5′ adjacent to the D element. Snyder et al., (1993) J. Virology 67:6096, identified the minimum trs sequence as 3′-GGT/TGA-5′, which substantially overlaps the sequence identified by Brister & Muzyczka.

The insertion can be of any suitable length that substantially reduces (e.g., by at least about 50%, 60%, 70%, 80%, 90%, 95%, 98% or greater) or eliminates resolution of the terminal repeat. The insertion can be at least about 3, 4, 5, 6, 10, 15, 20 or 30 nucleotides or more. There are no particular upper limits to the size of the inserted sequence, as long as suitable levels of viral replication and packaging are achieved (e.g., the insertion can be as long as 50, 100, 200 or 500 nucleotides or longer).

As another approach, the terminal repeat can be rendered non-resolvable by deletion of the trs site. The deletions may extend 1, 3, 5, 8, 10, 15, 20, 30 nucleotides or more beyond the trs site, as long as the template retains the desired functions. In addition to the trs site, some or all of the D element can be deleted (see, e.g., McCarty et al., (2003) Gene Therapy 10:2112-2118; McCarty et al., (2003) Gene Therapy 10:2112-2118; and WO 01/92551). Deletions can further extend into the A element; however those skilled in the art will appreciate that it may be advantageous to retain the RBE in the A element, e.g., to facilitate efficient packaging. Deletions into the A element can be 2, 3, 4, 5, 8, 10, or 15 nucleotides in length or more, as long as the non-resolvable terminal repeat retains any other desired functions: Further, some or all of the viral sequences going beyond the D element outside the terminal repeat sequence (e.g., to the right of the D element in FIG. 6 of PCT Publication No. WO 01/925551) can be deleted to reduce or prevent the process of gene conversion to correct the altered terminal repeat.

As still a further alternative, the sequence at the nicking site can be mutated so that resolution by Rep protein is reduced or substantially eliminated. For example, A and/or C bases can be substituted for G and/or T bases at or near the nicking site. The effects of substitutions at the terminal resolution site on Rep cleavage have been described by Brister & Muzyczka, (1999) J. Virology 73:9325. As a further alternative, nucleotide substitutions in the regions surrounding the nicking site, which have been postulated to form a stem-loop structure, can also be used to reduce Rep cleavage at the terminal resolution site (Id.).

Those skilled in the art will appreciate that the alterations in the non-resolvable terminal repeat can be selected so as to maintain desired functions, if any, of the altered terminal repeat (e.g., packaging, Rep recognition, and/or site-specific integration, and the like).

Further, the non-resolvable terminal repeat can be rendered resistant to the process of gene conversion as described by Samulski et al., (1983) Cell 33:135. Gene conversion at the non-resolvable terminal repeat will restore the trs site, which will generate a resolvable terminal repeat. Gene conversion results from homologous recombination between the resolvable terminal repeat and the altered terminal repeat.

One strategy to reduce gene conversion is to produce virus using a cell line (e.g., mammalian) that is defective for DNA repair, as known in the art, because these cell lines will be impaired in their ability to correct the mutations introduced into the viral template.

Alternatively, templates that have a substantially reduced rate of gene conversion can be generated by introducing a region of non-homology into the non-resolvable terminal repeat. Non-homology in the region surrounding the trs element between the non-resolvable terminal repeat and the unaltered terminal repeat on the template will reduce or even substantially eliminate gene conversion.

Any suitable insertion or deletion may be introduced into the non-resolvable terminal repeat to generate a region of non-homology, as long as gene conversion is reduced or substantially eliminated. Strategies that employ deletions to create non-homology are preferred. It is further preferred that the deletion does not unduly impair replication and packaging of the template. In the case of a deletion, the same deletion may suffice to impair resolution of the trs site as well as to reduce gene conversion.

As a further alternative, gene conversion may be reduced by insertions into the non-resolvable terminal repeat or, alternatively, into the A element between the RBE and the trs site. The insertion is typically at least about 3, 4, 5, 6, 10, 15, 20 or 30 nucleotides or more nucleotides in length. There is no particular upper limit to the size of the inserted sequence, which may be as long as 50, 100, 200 or 500 nucleotides or longer, however, generally, the insertion is selected so that it does not unduly impair replication and packaging of the vector genome.

Non-resolvable terminal repeats and duplexed parvovirus vectors are described in international patent publication WO 01/92551 and McCarty et al., (2003) Gene Therapy 10:2112-2118.

The virus vector can comprise any heterologous nucleotide sequence(s) of interest for delivery to connective tissue (including joint tissue). Nucleotide sequences of interest include nucleotide sequences encoding polypeptides, including therapeutic (e.g., for medical or veterinary uses) polypeptides.

Therapeutic polypeptides include, but are not limited to, growth factors (e.g., insulin-like growth factor (IGF)-I and/or -II), anti-catabolic factors (e.g., interleukin receptor antagonist protein (IRAP)), anti-inflammatory factors (e.g., TNF-α soluble receptor) and anti-oxidants (e.g., superoxide dismutase). Other exemplary therapeutic polypeptides include, but are not limited to, bone morphogenic proteins (e.g., BMP-2 and/or BMP-7), VEGF, RANKL, transforming growth factor beta (TGF-β) 1 and/or 2 (e.g., to alter commitment of cells to the chondrocyte or bone lineage), acidic fibroblast growth factor (FGF), basic FGF, TGFα, epidermal growth factor (EGF), vascular endothelial-derived growth factor, Sox9, and heparin binding growth factor.

In addition, the virus vector can be employed to deliver any heterologous nucleotide sequence with a biological effect to treat or ameliorate the symptoms associated with any connective tissue disorder related to gene expression. Illustrative connective tissue disorders include, but are not limited to: osteoarthritis (e.g., by administration of insulin-like growth factor I and/or II), partial or complete cartilage tears (e.g., by administration of insulin-like growth factor I and/or II), rheumatoid arthritis (e.g., by administration of anti-inflammatory factors such as IRAP and/or TNFα soluble receptor), bone fractures (e.g., by administration of bone morphogenic proteins [such as BMP-2 and/or BMP-7], VEGF and/or RANKL), and the like.

Heterologous nucleotide sequences encoding polypeptides include those encoding reporter polypeptides (e.g., an enzyme). Reporter polypeptides are known in the art and include, but are not limited to, green fluorescent protein (GFP), β-galactosidase, alkaline phosphatase, luciferase, and chloramphenicol acetyltransferase.

Alternatively, the heterologous nucleotide sequence may encode an antisense nucleic acid, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. No. 6,013,487; U.S. Pat. No. 6,083,702), interfering RNAs (RNAi) including siRNA and shRNA that mediate gene silencing (see, Sharp et al., (2000) Science 287:2431) or other non-translated RNAs, such as “guide” RNAs (Gorman et al., (˜1998) Proc. Nat. Acad. Sci. USA 95:4929; U.S. Pat. No. 5,869,248 to Yuan et al.), and the like. Exemplary untranslated RNAs include RNAi against IGF binding proteins (e.g., IGFBP 1, 2, 3, 4, 5 and/or 6, in particular, IGFBP3), TGF-β1 and/or 2 (e.g., to alter commitment of cells to the chondrocyte and/or bone lineage), and/or interleukin-1 receptor.

The virus vector can also comprise a nucleic acid that shares homology with and recombines with a locus on the host chromosome. This approach may be utilized to correct a genetic defect in the host cell.

Alternatively, the heterologous nucleotide sequence may encode any polypeptide that is desirably produced in a connective tissue cell in vitro, ex vivo, or in vivo. For example, the virus vector can be introduced into cultured cells and the expressed gene product isolated therefrom, or can be used to manipulate cells ex vivo, and the cells administered to a subject as a cell-based therapy, or the vector can be administered in vivo directly to a subject.

There are no particular size limits for the heterologous nucleotide sequence as long as it can be packaged and delivered by the virus vector. In general, the AAV capsid can efficiently package and deliver a vector genome that is approximately 80% to 105% of the wild-type AAV genome (4.68 kb), although larger recombinant vector genomes can be administered at reduced efficiency.

In particular embodiments, the heterologous nucleotide sequence is at least about 15, 18, 24, 50, 100, 250, 500, 1000 or more nucleotides long, and less than about 4.2 kb, 4.4 kb, 4.6 kb, 4.8 kb or 4.9 kb (with or without regulatory sequences). In the case of duplexed parvovirus vectors, both strands of the heterologous nucleotide sequence are delivered by the vector; accordingly, the heterologous nucleotide sequence will typically be less than about 2.1 kb, 2.2 kb, 2.3 kb, 2.4 kb or 2.45 kb in length (with or without regulatory sequences) to facilitate packaging of the duplexed sequence by the AAV capsid.

It will be understood by those skilled in the art that the heterologous nucleotide sequence(s) of interest can be operably associated with appropriate control sequences. For example, the heterologous nucleotide sequence may be operably associated with expression control elements, such as transcription/translation control signals, origins of replication, polyadenylation signals, internal ribosome entry sites (IRES), promoters, enhancers, and the like.

Those skilled in the art will appreciate that a variety of promoter/enhancer elements may be used depending on the level and tissue-specific expression desired. The promoter/enhancer may be constitutive or regulatable, depending on the pattern of expression desired. The promoter/enhancer may be native or foreign and can be a natural or a synthetic sequence. By foreign, it is intended that the transcriptional initiation region is not found in the wild-type host into which the transcriptional initiation region is introduced.

The promoter/enhancer element can be native to the target cell or subject to be treated and/or can be native to the heterologous nucleotide sequence. The promoter/enhancer element is generally chosen so that it will function in the target cell(s) of interest. The promoter/enhancer element can optionally be a mammalian promoter/enhancer element. The promoter/enhancer element may further be constitutive or regulatable (i.e., inducible).

Regulatable promoters/enhancer elements for nucleic acid delivery can be tissue-preferred and/or -specific promoter/enhancer elements, and include chondrocyte-preferred or -specific and synoviocyte-preferred or -specific promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein which the heterologous nucleotide sequence(s) are to be transcribed and then translated in the target cells, specific initiation signals may be included for efficient translation of inserted protein coding sequences. These exogenous translational control sequences, which may include the ATG initiation codon and adjacent sequences, can be of a variety of origins, both natural and synthetic.

The invention also encompasses the virus vectors of the invention, isolated cells that have been modified by introduction of the virus vectors therein, and pharmaceutical formulations comprising either of the foregoing.

### Methods of Nucleic Acid Delivery.

The present invention can be practiced to deliver a nucleic acid of interest to any connective tissue or connective tissue cell in vitro (including ex vivo) and in vivo. In particular embodiments, the connective tissue is cartilage, meniscus, synovium, bone and/or bone marrow. Optionally, the connective tissue is a joint tissue and/or a precursor to joint tissue (e.g., cartilage, meniscus, synovium and/or bone marrow). Non-limiting examples of joint tissue cells and precursors thereof include chondrocytes, synoviocytes, fibrocartilage cells, and/or bone marrow derived mesenchymal stem cells.

In the case of joint tissue, the nucleic acid can be delivered to the tissue of any suitable joint. For a human or non-human primate, the joint can be without limitation a knee joint, elbow joint, hip joint, ankle joint, shoulder joint, wrist joint, knuckle joint, or any combination thereof. In the case of a horse, cat or dog, the joint can be a carpus joint, elbow joint, hip joint, femoropatellar joint, scapulohumoral joint, femoral tibia joint, or any combination thereof.

Thus, as one aspect, the invention provides a method of delivering a nucleic acid to a connective tissue cell (e.g., a joint tissue cell or a precursor thereof), the method comprising contacting the cell with a virus vector comprising: (a) an AAV capsid; and (b) a recombinant nucleic acid comprising 5′ and 3′ AAV terminal repeats and a heterologous nucleotide sequence; wherein the recombinant nucleic acid is packaged within the AAV capsid. AAV vectors and heterologous nucleotide sequences of interest are as described herein.

In representative embodiments, the AAV capsid is an AAV2, AAV3 or AAV6 capsid and, optionally, the cell is a chondrocyte and/or a synoviocyte. As a further option, the virus vector can be a duplexed parvovirus vector as described herein.

According to this embodiment of the invention, the cell can be a cell in vitro (including a cell that is manipulated ex vivo) or in vivo. Methods of removing a cell from a subject or from a donor, introducing a virus vector therein ex vivo and then administering to a subject are known in the art, as are cell-based therapies for treating connective tissue disorders.

Thus, the invention also provides a method of delivering a nucleic acid to a connective tissue (e.g., a joint tissue or precursor thereof) of a subject, the method comprising administering to the subject a cell into which a virus vector has been introduced in vitro. In particular embodiments, the method comprises: (a) removing a cell from the subject; (b) introducing the virus vector into the cell and/or progeny thereof; and (c) administering the cell of (b) and/or progeny thereof to the subject. The cell can be without limitation a chondrocyte, synoviocyte, fibrocartilage cell, and/or a bone marrow derived mesenchymal stem cell. Alternatively, the cell can be from a donor subject. Non-limiting examples of connective tissue include cartilage, synovium, meniscus, bone and/or bone marrow. Optionally, the connective tissue is a joint tissue or a precursor to joint tissue (e.g., cartilage, meniscus, synovium and/or bone marrow).

The virus vectors of the invention can also be administered directly to the subject. In representative embodiments, the invention provides a method of administering a nucleic acid to a connective tissue (e.g., a joint tissue or a precursor thereof) in a subject, the method comprising administering to the subject a virus vector comprising: (a) an AAV capsid; and (b) a recombinant nucleic acid comprising 5′ and 3′ AAV terminal repeats and a heterologous nucleotide sequence; wherein the recombinant nucleic acid sequence is packaged within the AAV capsid. AAV vectors and heterologous nucleotide sequences of interest are as described herein.

In representative embodiments, the AAV capsid is an AAV2, AAV3 or AAV6 capsid and, optionally, the connective tissue is cartilage and/or synovium. As a further option, the virus vector can be a duplexed parvovirus vector as described herein.

The invention also encompasses methods of treating a connective tissue disorder (e.g., a joint disorder). In representative embodiments, the invention provides a cell-based method, of treating a connective tissue disorder (e.g., a joint disorder) comprising administering to a subject in need thereof an effective amount of a cell that has been transduced with a viral vector as described above. According to this aspect of the invention, the method can comprise: (a) removing a cell from the subject; (b) introducing the virus vector into the cell and/or a progeny thereof; and (c) administering the cell of (b) and/or a progeny thereof to the subject. Alternatively, the cell can be taken from a donor subject, the virus vector introduced into the cell and/or progeny thereof, and the cell and/or progeny thereof administered to the subject. As non-limiting examples, the cell can be a chondrocyte, a synoviocyte, fibrocartilage cells, and/or a bone marrow derived mesenchymal stem cell.

As another approach, the virus vector can be administered directly to the subject to treat a connective tissue disorder. Thus, the invention provides a method of treating a connective tissue disorder (e.g., a joint disorder) in a subject, the method comprising administering to a subject in need thereof an effective amount of a virus vector comprising: (a) an AAV capsid; and (b) a recombinant nucleic acid comprising 5′ and 3′ AAV terminal repeats and a heterologous nucleotide sequence; wherein the recombinant nucleic acid sequence is packaged within the AAV capsid.

According to the foregoing methods, the heterologous nucleotide sequence can encode any therapeutic polypeptide (e.g., a growth factor, an anticatabolic factor, or a combination thereof) or nontranslated RNA. Therapeutic polypeptides, growth factors, anticatabolic factors and untranslated RNA are as discussed herein. In particular embodiments, the heterologous nucleotide sequence encodes an insulin-like growth factor I and/or II, an interleukin receptor antagonist protein (IRAP), TGF-β, a bone morphogenic protein (e.g., BMP-2 and/or BMP-7), RANKL and/or VEGF or a combination thereof. Virus vectors are also as described herein. In particular embodiments, the AAV capsid is an AAV2, AAV3 or AAV6 capsid and, optionally, the connective tissue disorder is a cartilage disorder, a meniscus disorder and/or a synovium disorder. The virus vector can be a duplexed parvovirus vector, also as described herein.

The invention can be practiced to treat any connective tissue disorder including but not limited to a bone disorder and/or joint disorders (including cartilage disorders, synovium disorders and/or meniscus disorders). Exemplary connective tissue disorders include, but are not limited to bone fractures, joint inflammation, rheumatoid arthritis, osteoarthritis, a cartilage disorder (e.g., a partial or complete cartilage tear, a cartilage defect such as a degenerative injury and/or a mechanical injury [trauma], and/or cartilage injury due to an ACL ligament tear), a meniscus tear, a sports injury (e.g., an acute sports injury), hip dysplasia, or any combination of the foregoing.

As a non-limiting example, a nucleic acid encoding insulin-like growth factor I and/or II, TGF-β, a bone morphogenic protein (e.g., BMP-2 and/or BMP-7), VEGF and/or RANKL can be administered to a subject to treat a cartilage disorder (e.g., a partial or complete cartilage tear or a cartilage defect), osteoarthritis, or a combination thereof.

As another example, a nucleic acid encoding IRAP can be administered to a subject to treat rheumatoid arthritis and/or joint inflammation.

As yet a further example, a nucleic acid encoding a bone morphogenic protein (e.g., BMP-2 and/or BMP-7), VEGF and/or RANKL can be administered to treat a bone fracture.

In still other representative embodiments, bone marrow derived mesenchymal stem cells are removed from a patient, or obtained from a donor, and a virus vector is introduced therein. The virus vector can deliver any heterologous nucleotide sequence of interest, including without limitation IGF-I and/or 11, TGF-β, bone morphogenic protein (BMP-2 and/or BMP-7), VEGF, RANKL and/or Sox9. The modified cell (or progeny thereof) can be implanted into a bone defect (e.g., a fracture) or a cartilage defect, where it can differentiate into a bone cell or chondrocyte, respectively.

The invention also encompasses a method of enhancing cartilage healing and/or regeneration. To illustrate, in particular embodiments, the invention provides a cell-based method of enhancing cartilage healing and/or regeneration, the method comprising administering to a subject in need thereof an effective amount of a cell that has been transduced as described above with a viral vector that delivers a nucleic acid encoding a growth factor (e.g., insulin like growth factor I). According to this aspect of the invention, the method can comprise: (a) removing a cell from the subject; (b) introducing the virus vector into the cell and/or progeny thereof; and (c) administering the cell of (b) and/or progeny thereof to the subject. Alternatively, the cell can be taken from a donor subject, the virus vector introduced therein, and the cell and/or progeny thereof administered to the subject. As non-limiting examples, the cell can be a chondrocyte, a synoviocyte, a fibrocartilage cell, and/or a bone marrow derived mesenchymal stem cell. In particular embodiments, the virus vector comprises an AAV2, AAV3 or AAV6 capsid and/or the virus vector is a duplexed parvovirus vector.

According to other embodiments, the invention provides a method of enhancing cartilage healing and/or regeneration, the method comprising administering to a subject in need thereof an effective amount of a virus vector comprising: (a) an AAV capsid; and (b) a recombinant nucleic acid comprising 5′ and 3′ AAV terminal repeats and a heterologous nucleotide sequence encoding a growth factor (e.g., insulin like growth factor I); wherein the recombinant nucleic acid sequence is packaged within the AAV capsid. In particular embodiments, the virus vector comprises an AAV2, AAV3 or AAV6 capsid and/or the virus vector is a duplexed parvovirus vector.

By “enhance,” “enhances,” “enhancing” cartilage healing and/or regeneration (and grammatical variations thereof) is meant an increase and/or acceleration in cartilage healing and/or regeneration that is of some benefit to the subject, e.g., at least about a 25%, 50%, 70%, 85%, 100%, 200%, 300%, 500% or more increase and/or acceleration.

The invention further provides a method of reducing joint inflammation (e.g., in cartilage and/or synovium). To illustrate, in particular embodiments, the invention provides a cell-based method of reducing joint inflammation, the method comprising administering to a subject in need thereof an effective amount of a cell that has been transduced with a viral vector that delivers a nucleic acid encoding an anti-inflammatory factor and/or anticatabolic factor (e.g., IRAP and/or TNF-α soluble receptor) as described above. According to this aspect of the invention, the method can comprise: (a) removing a cell from the subject; (b) introducing the virus vector into the cell and/or progeny thereof; and (c) administering the cell of (b) and/or progeny thereof to the subject. Alternatively, the cell can be taken from a donor subject, the virus vector introduced therein, and the cell and/or progeny thereof administered to the subject. As non-limiting examples, the cell can be a chondrocyte, a synoviocyte, a fibrocartilage cell, and/or a bone marrow derived mesenchymal stem cell. In particular embodiments, the virus vector comprises an AAV2, AAV3 or AAV6 capsid and/or the virus vector is a duplexed parvovirus vector.

The invention also provides a method of reducing joint inflammation in a subject, the method comprising administering to a subject in need thereof an effective amount of a virus vector comprising: (a) an AAV capsid; and (b) a recombinant nucleic acid comprising 5′ and 3′ AAV terminal repeats and a heterologous nucleotide sequence encoding an anti-inflammatory factor and/or anticatabolic factor (e.g., IRAP and/or TNF-α soluble receptor); wherein the recombinant nucleic acid sequence is packaged within the AAV capsid. In particular embodiments, the virus vector comprises an AAV2, AAV3 or AAV6 capsid and/or the virus vector is a duplexed parvovirus vector.

By “reduce,” “reduces,” or “reducing” joint inflammation (and grammatical variations thereof) is meant a decrease and/or delay and/or prevention of joint inflammation, e.g., at least about a 25%, 35%, 50%, 60%, 70%, 80%, 90%, 95% or more decrease and/or delay and/or prevention of joint inflammation.

The present invention finds use in research as well as veterinary and medical applications. Suitable subjects are generally mammalian subjects. The term “mammal” as used herein includes, but is not limited to, humans, non-human primates, cattle, sheep, goats, pigs, horses, cats, dog, rabbits, rodents (e.g., rats or mice), etc. Human subjects include neonates, infants, juveniles, adults and geriatric subjects.

In particular embodiments, the subject is a human or animal subject (e.g., a horse, dog or cat) that has or is at risk for a connective tissue disorder and is “in need of” the methods of the present invention, e.g., in need of the therapeutic and/or prophylactic effects of the inventive methods. For example, the subject can have or be at risk for a connective tissue disorder selected from the group consisting of a bone fracture, joint inflammation, rheumatoid arthritis, osteoarthritis, a cartilage disorder (e.g., a partial or complete cartilage tear or a cartilage defect such as a degenerative injury and/or a mechanical injury [trauma] and/or a cartilage injury following an ACL ligament tear), a meniscus tear, a sports injury, and any combination thereof.

In other embodiments, the subject used in the methods of the invention is an animal model of a connective tissue disorder (e.g., a rat, mouse, rabbit, horse, goat, sheep, dog or pig).

### Pharmaceutical Formulations and Delivery Routes.

In particular embodiments, the present invention provides a pharmaceutical composition comprising a virus particle or cell of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid.

By “pharmaceutically acceptable” it is meant a material that (i) is compatible with the other ingredients of the composition without rendering the composition unsuitable for its intended purpose, and (ii) is suitable for use with subjects as provided herein without significant undue adverse side effects (such as toxicity, irritation, and allergic response). Side effects are “undue” when their risk outweighs the benefit provided by the composition. Examples of pharmaceutically acceptable carriers include, without limitation, any of the standard pharmaceutical carriers such as phosphate buffered saline solutions, water, emulsions such as oil/water emulsions, microemulsions, and the like A pharmaceutically acceptable carrier or composition of this invention can also be a sterile carrier or composition.

One aspect of the present invention is a method of transferring a virus vector comprising a nucleotide sequence to a cell in vitro (including ex vivo). The virus particles may be introduced into the cells at the appropriate multiplicity of infection according to standard transduction methods appropriate for the particular target cells. Titers of virus to administer can vary, depending upon the target cell type and number, and the particular virus vector, and can be determined by those of skill in the art without undue experimentation. In particular embodiments, the cell is contacted with at least about 5×10² or 10³ transducing units, or even at least about 10⁴ 10⁵ 10⁶ or 10⁷ transducing units.

The cell can be any connective tissue cell including without limitation a chondrocyte, a synoviocyte, a fibrocartilage cell, a bone cell, or a bone marrow derived mesenchymal stem cell. Moreover, the cell can be from any species of origin, as described herein. In particular embodiments, the cell is from a human or from a horse, cat, dog, rabbit, rat, mouse, goat, sheep or pig.

In representative embodiments, the cell is a chondrocyte or synoviocyte and the virus vector comprises an AAV2, AAV3 or AAV6 capsid. Optionally, the virus vector can further be a duplexed parvovirus vector.

The virus vector can be introduced into cells in vitro for the purpose of administering the modified cell to a subject. For example, the cell can be removed from a subject, the virus vector can be introduced into the cell and/or progeny thereof, and the cell and/or progeny thereof can then be replaced back into the subject. Methods of removing cells from a subject for expansion and/or for manipulation ex vivo, followed by introduction back into the subject are known in the art. For example, Genzyme Biosurgery offers Carticel®, a commercial process to culture a subject's own chondrocytes for use in the repair of symptomatic cartilage defects. Alternatively, the virus vector can be introduced into cells from a donor subject, into cultured cells, or into cells from any other suitable source, and the cells and/or progeny thereof can be administered to a subject in need thereof.

Suitable connective tissue cells for ex vivo manipulation are as described above. Dosages of the cells to administer to a subject will vary upon the age, condition and species of the subject, the type of cell, the nucleic acid being expressed by the cell, the mode of administration, and the like. Typically, at least about 10³, 10⁴, 10⁵, 10⁶, 10⁷ or 10⁸ to about 10⁵, 10⁶, 10⁷ 10⁸, 10⁹ or 10¹⁰ cells can be administered per dose (in any combination satisfying the condition that the lower limit is less than the upper limit) in a pharmaceutically acceptable carrier. In particular embodiments, the cells transduced with the virus vector and/or progeny thereof are administered to the subject in a treatment effective amount in combination with a pharmaceutical carrier.

In particular embodiments, an effective dosage of a duplexed parvovirus vector is reduced by at least about 10-fold, 50-fold, 100-fold, 500-fold, 1000-fold or more with respect to a comparable non-duplexed rAAV vector.

A further aspect of the invention is a method of administering the virus vectors of the invention directly to a subject. Administration of the virus vectors to a human subject or an animal can be by any means known in the art for administering virus vectors. In particular embodiments, the virus vector is delivered in a treatment effective dose in a pharmaceutically acceptable carrier. Methods of preparing such dosage forms are known, or will be apparent, to those skilled in this art; for example, see Remington's Pharmaceutical Sciences, Mack Publishing Company, Easton, Pa., latest edition.

Dosages of the virus vector to be administered to a subject will depend upon the mode of administration, the disease or condition to be treated, the individual subject's condition, the particular virus vector, and the nucleic acid to be delivered, and can be determined in a routine manner. Exemplary doses are dosages of at least about 10⁵, 10⁶, 10⁷, 10⁸, 10⁹, 10¹⁰, 10¹¹, 10¹², 10³, 10¹⁴ or 10¹⁵ transducing units or more, for example, from about 10⁶, 10⁷ or 10⁸ to 10¹⁰, 10¹¹, 10¹² or 10¹³ transducing units.

In particular embodiments, an effective dosage of a duplexed parvovirus vector is reduced by at least about 10-fold, 50-fold, 100-fold, 500-fold, 1000-fold or more with respect to a comparable non-duplexed rAAV vector.

According to some embodiments, the virus vector comprises an AAV2, AAV3 or AAV6 capsid and is used to deliver a nucleic acid of interest to cartilage and/or synovium. Optionally, the virus vector is a duplexed parvovirus vector.

In particular embodiments, a virus vector comprising an AAV2, AAV3 or AAV6 capsid and comprising a heterologous nucleotide sequence encoding a growth factor (e.g., IGF-I and/or IGF-II), TGF-β, a bone morphogenic protein (e.g., BMP-2 and/or BMP-7), VEGF and/or RANKL is administered to enhance cartilage healing and/or regeneration, to treat a cartilage disorder and/or to treat osteoarthritis. Optionally, the virus vector is a duplexed parvovirus vector.

Further, in other exemplary embodiments, a virus vector comprising an AAV2, AAV3 or AAV6 capsid and comprising a heterologous nucleotide sequence encoding an anti-catabolic factor is administered to reduce inflammation and/or to treat osteoarthritis. Optionally, the virus vector is a duplexed parvovirus vector.

According to the methods of the invention, more than one administration (e.g., two, three, four or more administrations) may be employed to achieve the desired level of gene product over a period of various intervals, e.g., daily, weekly, monthly, yearly, etc.

The methods of the invention can be practiced in combination with any other suitable therapy for treating a connective tissue disorder.

The virus vectors, cells and pharmaceutical formulations of the invention can be administered by any method known in the art, including but not limited to direct injection, for example, direct injection into the joint (e.g., into the cartilage, synovium, meniscus and/or bone marrow), intravenous administration (optionally, with a vector that is modified to be targeted to the target cells of interest), or by application of a biomaterial (e.g., a collagen gel) containing the vector, cell or pharmaceutical formulation (e.g., implantation to fill in a bone defect such as a fracture or a cartilage or meniscus defect such as a cartilage tear or meniscus tear). As another example, one can administer the virus or cell in a depot or sustained-release formulation. Further, virus vectors can be delivered dried to a surgically implantable matrix such as a bone graft or bone graft substitute, a suture or other surgically implantable material (e.g., as described in U.S. Patent Publication No. US-2004-0013645-A1).

The most suitable route in any given case can be routinely determined and will depend on a variety of factors including the nature and severity of the condition being treated, the size, species and condition of the subject, and on the nature of the particular vector that is being used.

Direct administration to a connective tissue (e.g., direct administration to a joint or to bone) can be achieved by any suitable method known in the art, e.g., by injection into or near the connective tissue, joint or bone or by implantation of cells, for example, in combination with a biomaterial or any other method of local administration such as administration of virus vector dried to a surgically implantable matrix as described in U.S. Patent Publication No. US-2004-0013645-A1.

In particular embodiments, the virus vector or cells of the invention can be implanted into a connective tissue (for example, into a bone fracture, a meniscus tear or a cartilage tear or other defect) in combination with a biomaterial. For example, virus vector or cells that have been transduced with virus vector can be combined with a biomaterial such as a polymer (e.g., a collagen gel or a fibrin glue) and the biomaterial in combination with the virus vector or cells used to fill in a defect in bone, cartilage, meniscus, synovium and the like.

This aspect of the invention can be practiced with a wide variety of biocompatible matrices. Optionally, the matrix is biodegradable. Suitable biodegradable matrices are well known in the art and include without limitation collagen-GAG, collagen, fibrin, polylactic acid (PLA), polyglycolic acid (PGA), and PLA-PGA co-polymers. Additional biodegradable materials include poly(anhydrides), poly(hydroxy acids), poly(ortho esters), poly(propylfumerates), poly(caprolactones), polyamides, polyamino acids, polyacetals, biodegradable polycyanoacrylates, biodegradable polyurethanes and polysaccharides. Non-biodegradable polymers may also be used as well. For example, polypyrrole, polyanilines, polythiophene, and derivatives thereof are useful electrically conductive polymers that can provide additional stimulation to implanted cells. Other non-biodegradable, yet biocompatible polymers include polystyrene, polyesters, non-biodegradable polyurethanes, polyureas, poly(ethylene vinyl acetate), polypropylene, polymethacrylate, polyethylene, polycarbonates, and poly(ethylene oxide). Those skilled in the art will recognize that this is an exemplary, not a comprehensive, list of polymers suitable for the practice of the present invention.

The virus vector and/or cells can also be administered directly (e.g., by injection or by implantation) in combination with a water-soluble biocompatible gel, for example as described in U.S. Patent Publication 2006/0078542 A1 (Mah et al.). The biocompatible gel can comprise a sol, a matrix, a biogel, a hydrogel, a polymer, a polysaccharide, an oligosaccharide, or a viscous suspension, which may optionally be cross-linked, stabilized, chemically conjugated, or otherwise modified. The biocompatible gel can comprise one or more of the commercially-available gel compounds including, for example, alginate hydrogels SAF-Gel (ConvaTec, Princeton, N.J.), Duoderm Hydroactive Gel (ConvaTec), Nu-gel (Johnson & Johnson Medical, Arlington, Tex.); Carrasyn (V) Acemannan Hydrogel (Carrington Laboratories, Inc., Irving, Tex.); glycerin gels Elta Hydrogel (Swiss-American Products, Inc., Dallas, Tex.), K-Y Sterile (Johnson & Johnson), or combinations thereof.

Pharmaceutical compositions suitable for parenteral administration can comprise sterile aqueous and non-aqueous injection solutions of the composition of this invention, which preparations are preferably isotonic with the blood and/or other body fluids of the intended recipient. These preparations can contain anti-oxidants, buffers, bacteriostats and solutes, which render the composition isotonic with the blood and/or other body fluids of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Injectables can be prepared in conventional forms, either as liquid solutions or suspensions, solid forms suitable for solution or suspension in liquid prior to injection, or as emulsions. Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Having described the present invention, the same will be explained in greater detail in the following examples, which are included herein for illustration purposes only, and which are not intended to be limiting to the invention.

### EXAMPLES

### Materials & Methods

### Cell Culture Preparation & Transduction:

Equine chondrocytes and synoviocytes were harvested from the femoropatellar and scapulohumoral joints of immature foals. Joints were digested in 0.75 mg collagenase (Worthington Biochemicals) per ml of medium. After digestion of tissues, cells were counted and stored in liquid nitrogen. Cell monolayers were seeded at 50% confluency density in HEPES-buffered Ham's F12 medium (GIBCO, Grand Island, N.Y.) with L-glutamine (300 μg/ml), α-ketoglutaric acid (30 μg/ml), and 10% fetal bovine serum (FBS). Cultures were grown in duplicate in 24-well plates (Corning Inc, Corning, N.Y.).

### Initial Screening of AAV Serotypes:

Two days after seeding, cells were washed with serum-free medium. Cells were then transduced with self-complementary rAAV serotype 1, 2, 3 (serotype 3B), 4, 6 or 8 vector delivering a cytomegalovirus (CMV) driven green fluorescent protein (GFP) construct at 10000, 1000, 100, and 10 virus particles/cell or with rAAV serotype 5, which had the lowest titer, at 1000, 100, 10, and 1 virus particles/cell. Cells were allowed to uptake virus for 4 hours at 37° C. in the serum-free minimal essential medium (GIBCO, Grand Island, N.Y.) and then nourished with the supplemented Ham's F12 growth medium described above. Growth medium was replaced every 48 hr. Fluorescence was measured daily using a SPECTRAmax GEMINI-XS fluorometer with excitation at 472 nm, emission 512 nm, and a 495 nm emission cutoff filter. The entire well surface was scanned using 10 reads/well. GFP fluorescence was also monitored using an Olympus IX70 fluorescence microscope with a 20× optical plus a 1.5× extra zoom and a WU filter that has an excitation from 330-385 nm and an emission >420 nm. Images were obtained with this microscope and BioQuant software every day for 90 days to monitor how long the cells fluoresced.

### Optimization and Dose Response of Select AA V Serotypes:

From the initial screening of the serotypes, four optimal AAV serotypes (serotypes 2, 3, 5 and 6) were used for further optimization and dose response. Serotypes 2, 3 and 6 were transduced at 8000, 4000, 2000, 1000 and 500 virus particles/cell into chondrocytes and synoviocytes 2 days after seeding. Serotype 5 was transduced at 4000, 2000, 1000, and 500 virus particles/cell. Cells were monitored for fluorescence with the above methods daily. Viability of transduced cells was determined when cells reached >100% confluency and counting total number of live cells versus number of dead cells using trypan blue (GIBCO, Grand Island, N.Y.) staining.

### Toxicity of Serotypes on Synoviocytes and Chondrocytes:

Toxicity of the AAV serotypes was measured by quantitative PCR and relative gene expression of equine MMP-1, MMP-3, MMP-13 and aggrecanase. Expression of all genes was compared to the expression levels of equine GAPDH. RNA was extracted from cells using the RNEASY mini kit (Qiagen, Valencia, Calif.). Complementary DNA was generated using 50 ng of total RNA and oligo(dT) primers with the SUPERSCRIPT III first-strand synthesis kit (Invitrogen, Carlsbad, Calif.). Equine MMP-1, MMP-3, MMP-13, and GAPDH primer/probes were obtained from the Lucy Whittier Molecular Core Facility (UC Davis, Davis, Calif.). Aggrecanase sequences were as follows: forward primer 5′-GCCTTCACTGCTGCTCATGA-3′ (SEQ ID NO:1), reverse primer 5′-CCAACACATGGCTTTGAATTGT-3′ (SEQ ID NO:2), and probe 5′-FAM-CTGGGCCATGTCTTCAACATGCTCC-TAMRA-3′ (SEQ ID NO:3). Quantitative PCR was run using 2.5 ng RNA per sample (in duplicate) on an ABI Prism 7000 (Applied Biosystems, Foster City, Calif.) with a 50° C., 2 minute hold; a 95° C., 10 minute denaturation; followed by 40 cycles of a 95° C., 15 second denaturation and a 60° C., 30 second anneal.

### Results

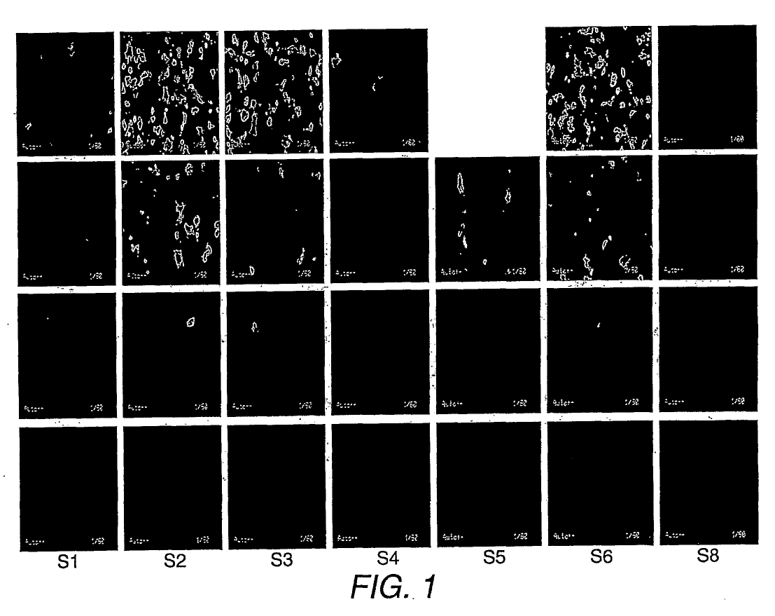

FIG. 1 shows a fluorescence photomicrograph of chondrocytes at day 7 following transduction with AAV-GFP serotype 1, 2, 3, 4, 5, 6 or 8. The top row represents 10,000 viral particles per cell, the second row 1000, third row 100, and bottom row 10 viral particles per cell. The optimal serotypes for chondrocytes from this initial screening were AAV-GFP serotypes 2, 3, 5 and 6.

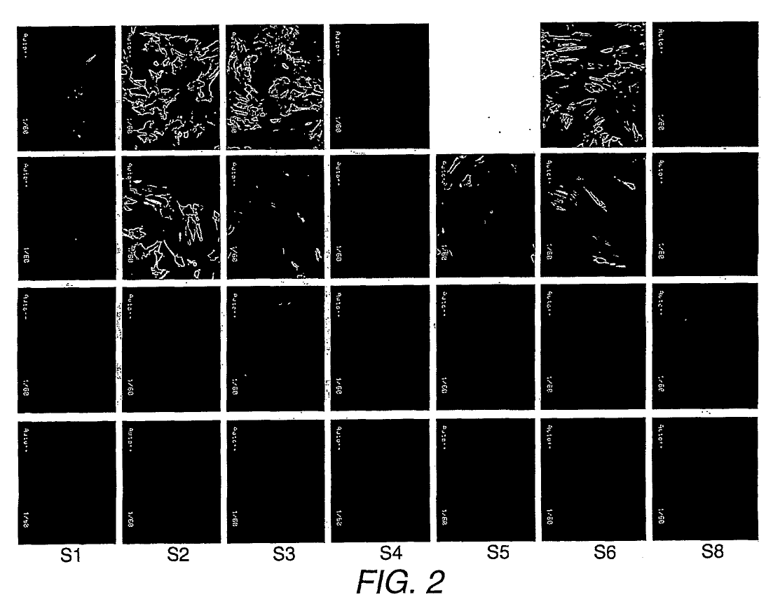

FIG. 2 shows a fluorescence photomicrograph of synoviocytes at day 7 following transduction with AAV-GFP serotype 1, 2, 3, 4, 5, 6 or 8. The top row represents 10,000 viral particles per cell, the second row 1000, third row 100, and bottom row 10 viral particles per cell. The optimal serotypes for synoviocytes from this initial screening were AAV-GFP serotypes 2, 3, 5 and 6.

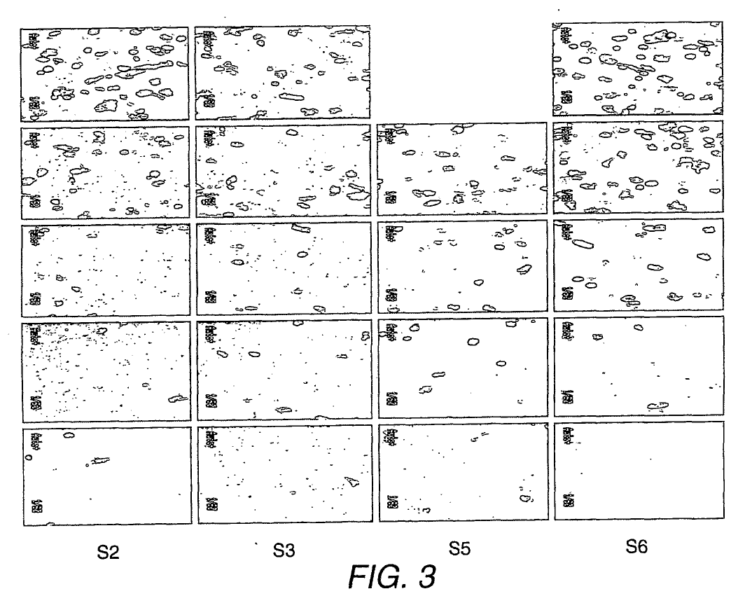

FIG. 3 shows a fluorescence photomicrograph representing the second set of chondrocyte transductions for AAV-GFP serotypes 2, 3, 5 and 6. The top row represents 8000 viral particles per cell, second row 4000, third row 2000, fourth row 1000, and bottom row 500. Serotypes 2 and 6 demonstrated optimal transduction with minimal differences in efficiencies between serotypes 2 and 6. However the cellular morphology for chondrocytes transduced with serotype 2 revealed rounded and crenated cell types, a characteristic that is atypical for chondrocytes in monolayer. From the gross morphology, AAV-GFP serotype 6 appears to be an optimal serotype for equine chondrocytes with little variation in transduction efficiency between 8000 and 4000 viral particles per cell.

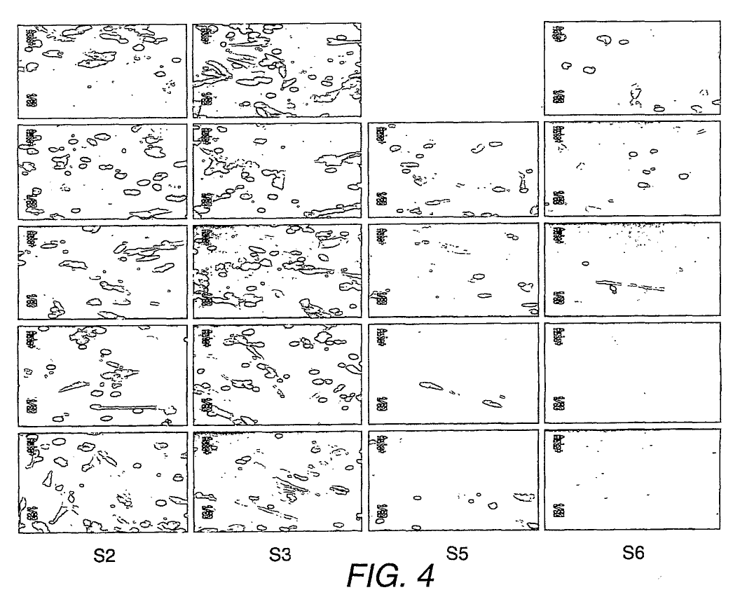

FIG. 4 shows a fluorescence photomicrograph representing the second set of synoviocyte transductions for AAV-GFP serotypes 2, 3, 5 and 6. The top row represents 8000 viral particles per cell, second row 4000, third row 2000, fourth row 1000, and bottom row 500. Serotypes 2 and 3 demonstrated optimal transduction with minimal differences in efficiencies between serotypes 2 and 3. However the cellular morphology for synoviocytes transduced with serotype 2 revealed rounded and crenated cell types, a characteristic that is atypical for synoviocytes in monolayer. From the gross morphology AAV-GFP serotype 3 appears to be an optimal serotype for equine synoviocytes with little variation in transduction efficiency between 8000 and 4000 viral particles per cell.

FIG. 5 represents transduction efficiencies for different AAV serotypes in chondrocytes (top graph) and synoviocytes (bottom graph) up to passage 3 (day 51). Transduction efficiencies for the 4 serotypes (2, 3, 5 and 6) in chondrocytes ranged from 48% to 85% on day 3. Transduction efficiencies rose at day 7 and then dropped at day 17 to between 38% (serotype 5) and 65% (serotype 6). Transduction efficiencies continued to range between 30 and 50 percent through passage 2. At this time (day 35) cell populations only consisted of approximately 25% of the original cells due to cell expansion and passage.

Transduction efficiency for synoviocytes ranged between 38% (serotype 5) and 82% (serotypes 2 and 3). Transduction efficiencies rose until day 7 and dropped at day 17 (passage 1). Serotype 3 appeared to maintain the optimal transduction efficiency until passage 3. Similar to chondrocytes, at day 51 cell populations only consisted of approximately 25% of the original cells due to cell expansion and passage.

FIG. 6 represents the relative fluorescence measured with the fluorometer from day 1 through day 7 for AAV-GFP serotypes 2, 3, 5 and 6 at 4000 viral particles per cell for all serotypes. Serotype 6 demonstrated optimal fluorescence relative to serotypes 2, 5 and 6. Relative fluorescence in synoviocytes (bottom panel) revealed that serotypes 2 and 3 were comparable to one another through day 4, and at day 7, serotype 3 had improved fluorescence over serotype 2.

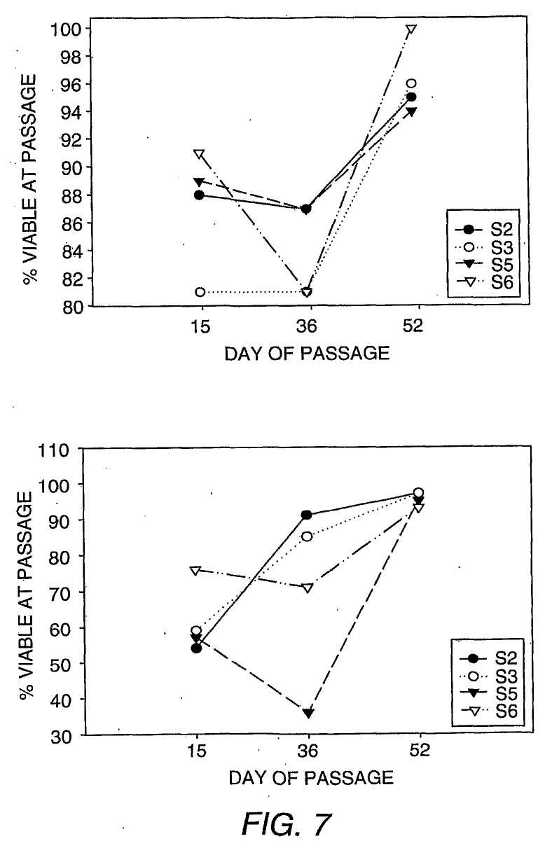

FIG. 7 represents cell viability for chondrocytes (top panel) and synoviocytes (bottom panel). Cell viability was determined at passages 1 (day 15), 2 (day 36) and 3 (day 52). Cell viability for chondrocytes was optimal for serotype 6 at passages 1 and 3. Cell viability for synoviocytes was optimal for serotype 6 at passage 1 (day 15).

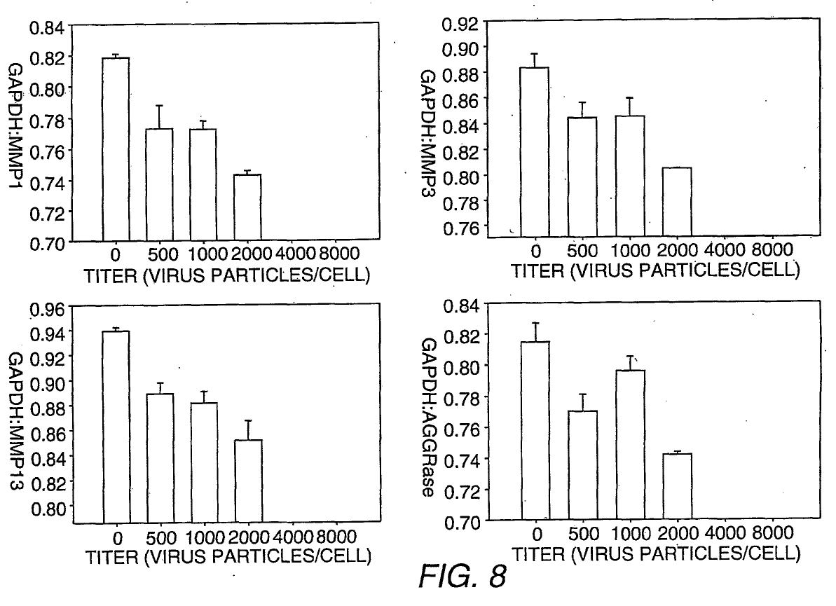

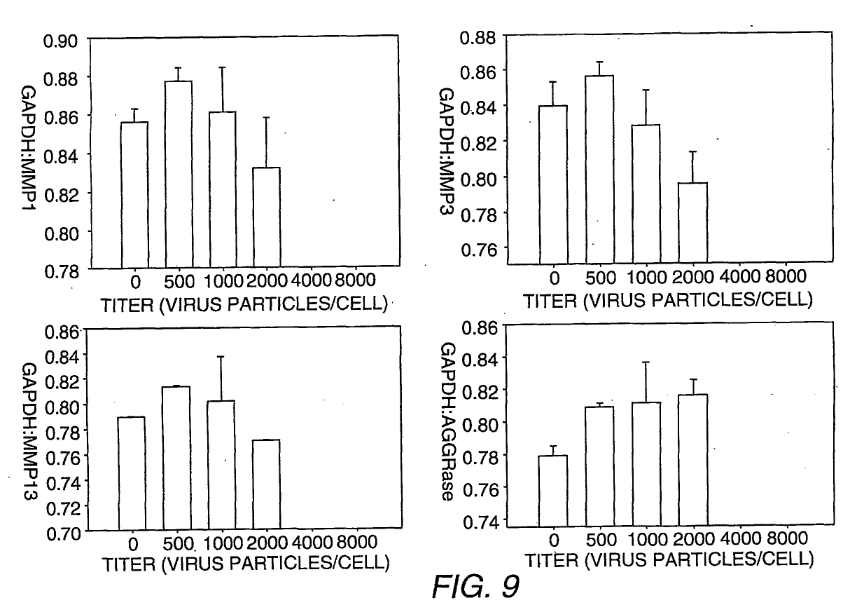

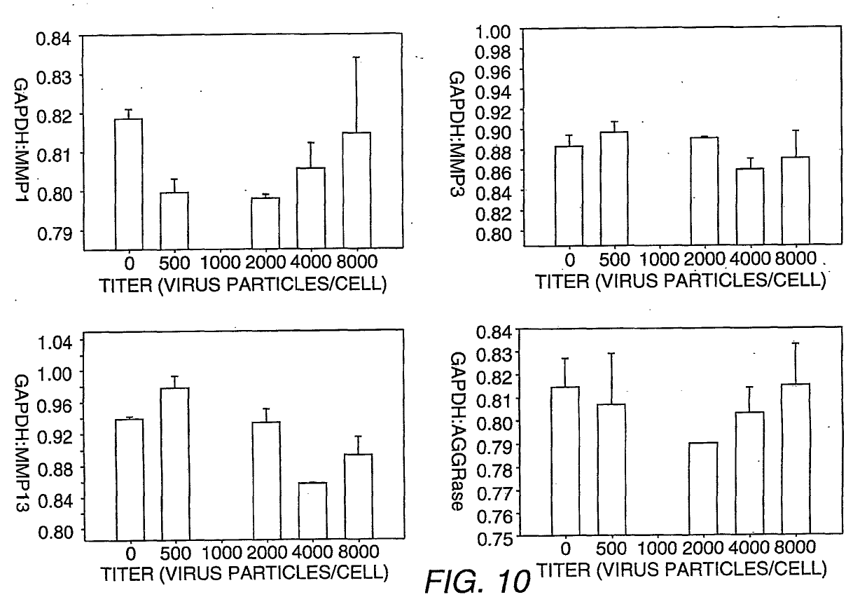

FIGS. 8 and 9 show RNA expression profiles for inflammatory molecules for chondrocytes transduced with serotype 6 (FIG. 8) and synoviocytes transduced with serotype 3 (FIG. 9). Minimal differences existed for doses 0 through 2000 viral particles per cell (not enough RNA was harvested for titers 4000 and 8000). FIG. 10 demonstrates serotype 2 MMP1 RNA expression in chondrocytes (top, left graph) and suggests a slight increase in MMP1 for serotype 2 at increasing viral titers. RNA expression profiles for MMP3, MMP13 and aggrecanase 1 did not show any difference with serotype.

scAAV-IGF-I Materials and Methods

Chondrocytes and synoviocytes from equine stifle joints were cultured in duplicate in 48-well culture dishes at 50% confluency. Chondrocytes were transduced with serotype 6 (S6) scAAV-insulin growth factor-I (scAAV-IGF-1) at 4000 particles/cell and the synoviocytes were transduced with serotype 3 (S3) scAAV-IGF-I at 4000 particles/cell. Cells were grown in monolayer for three days post transduction prior to trypsinizing followed by culture in alginate. A portion of cells were lifted and re-plated in monolayer to see if growth in alginate affected IGF production.

For alginate casting, cells were lifted from the monolayer and the cell pellet was resuspended in 20 μl 1×PBS with 80 μl 1.2% alginate in PBS. The alginate mixture was added dropwise to 102 mM CaCl₂ to form beads. The CaCl₂ was removed and replaced with F-12 medium ((GIBCO, Grand Island, N.Y.) with L-glutamine (300 μl/ml), α-ketoglutaric acid (30 μl/ml), 25 mM HEPES (GIBCO, Grand Island, N.Y.), and 10% fetal bovine serum (FBS, HyClone, Logan, Utah)). Medium was collected on days 3, 7, 10, and 14 days post transduction. Medium was subsequently assayed for IGF production with R&D systems quantikine human IGF sandwich ELISA kit.

To determine the optimal alginate concentration for cell growth and IGF production, cells were transduced with the above serotypes of scAAV-IGF-I and grown in 1.2, 1.0, 0.8, and 0.6% alginate in duplicate. Cells were grown for 3 days and counted prior to lifting and culturing in alginate. This was considered day 0. Cells were harvested from the alginate matrix by removing growth medium and replacing it with alginate digestion buffer (55 mM Na citrate, 30 mM EDTA, 150 mM NaCl) for 5-10 minutes. Cells were resuspended in this buffer, centrifuged at 8000×g for 5 minutes, buffer aspirated, and the cell pellet resuspended in 500 μl PBS. An aliquot was removed for staining and counting with Trypan Blue. Medium was collected at 7 and 14 days and growth was monitored at 3, 7, 10, and 14 days post transduction.

### Results

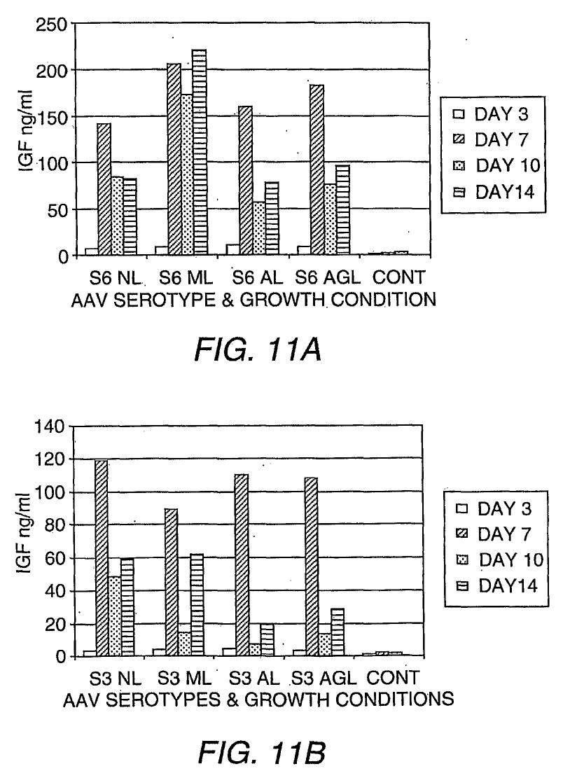

Monolayer cultures of chondrocytes (FIG. 11A) and synoviocytes (FIG. 11B) had the highest IGF production when compared to alginate cultures over the 14 day time period. The reduced IGF production in alginate cultures could be due to the alginate concentration.

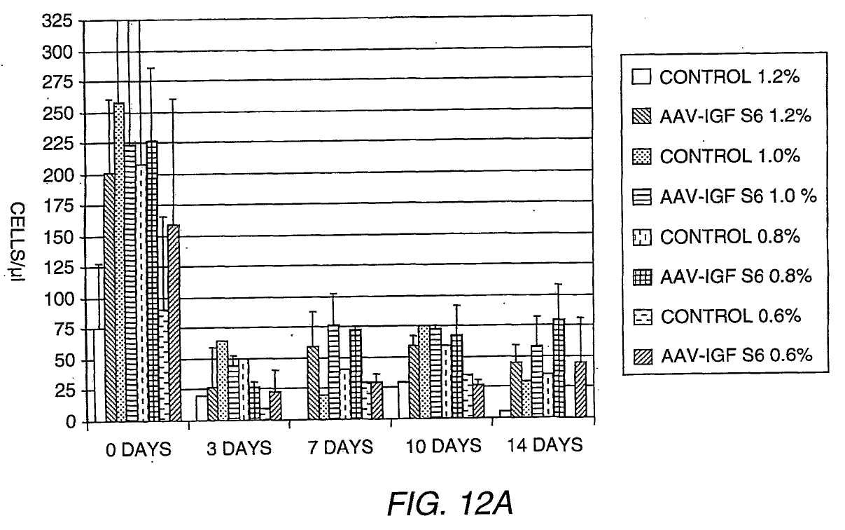

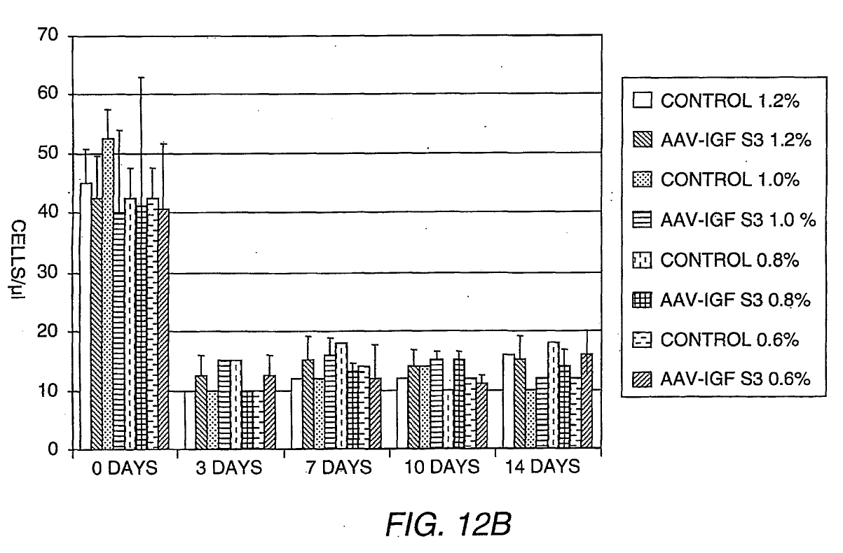

Chondrocytes (FIG. 12A) and synoviocytes (FIG. 12B) had the most growth at day 0 prior to being cultured in alginate. Over time, the 0.8% alginate seemed to have more cell growth than the other alginate constructs.

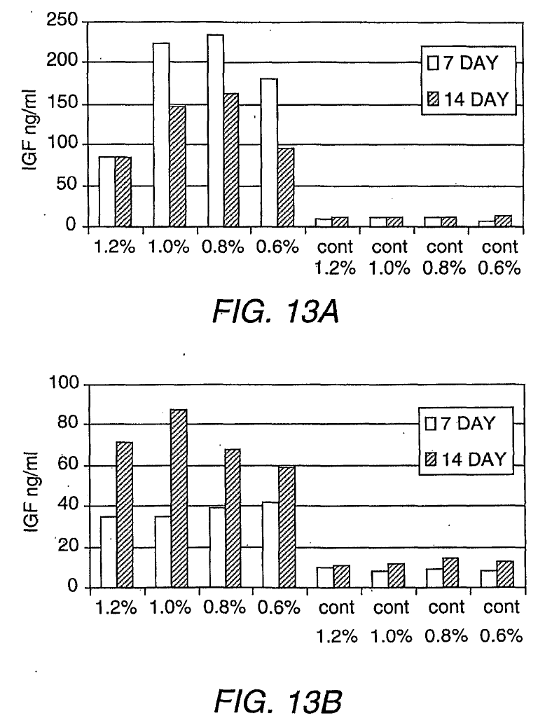

For chondrocytes, the highest production of IGF occurred at day 7 in 0.8% alginate (FIG. 13A). Synoviocytes had the highest production of IGF at day 14 in 1.0% alginate (FIG. 13B). IGF production was high at 0.6% alginate on day 7 for both cell types.

### AAV-GFP Materials & Methods

Determination of Transduction Efficiency with Flow Cytometry.

From the second screening of the serotypes, four optimal AAV serotypes (S2, S3, S5, and S6) were used for selection of GFP cell populations using flow cytometry. Chondrocytes and synoviocytes from four foals were seeded in duplicate. S2, S3, S5, and S6 were transduced at 4000, 2000, and 1000 particles/cell when cells were 50% confluent. Transduction efficiency was measured by monitoring GFP fluorescence by fluorometer readings, hemacytometer counts, and flow cytometry. Fluorometer readings were obtained on days 3, 7, 10, and 14. Cells were treated with 0.25% trypsin to lift cells from the monolayer, resuspended in 500 μl growth medium, and replicate wells were combined into 1 microfuge tube for each condition for sorting by flow cytometry on days 7 and 14. Sorting was conducted on a MoFlo Cell Sorter/Analyzer (Dako, Fort Collins, Colo.) with a 100 micron flow cell tip used at 30 psi sheath pressure and a flow rate of 12,000 events per second and laser line of 488 nm with laser power of 110. Cells were sorted based on GFP fluorescence with a 530/540 band pass filter, preceded by a neutral density 2.0 filter and high voltage of 400-450 with a log signal. SUMMIT software (version 4.0, Dako, Fort Collins, Colo.) was used to collect histograms and set sort parameters. Hemacytometer counts were obtained on days 7 and 14. Briefly, 50 μl of cell suspension was mixed with an equal volume of Trypan Blue, cells were placed on a hemacytometer, and the number of live, dead, and fluorescing cells was counted. After sorting, 80% of the cell population, determined through hemacytometer counts, was reserved for RNA isolation while 20% of the cell population was replated into one well of a 24-well plate for further monitoring of fluorescence up to day 14.

### Morphological Effects of AAV Serotypes:

Cells were scored on days 0, 3, 6, 10 and 14 following transduction with AAV serotypes 2, 3, 5 and 6. Cells were given a score of 0 through 5 based on morphological characteristics. Abnormal morphological characteristics were considered to be rounding, crenation or lifting off of the plate. Scoring was as follows. 1=1-20% of the cells had abnormal cell morphology, 2=21-40, 3=41-60, 4=61-80, and 5=81-100% abnormal. Five views at 200× magnification were assessed and an average score was determined.

Assessment of Inflammatory Molecules using Quantitative PCR:

Toxicity of the AAV serotypes was measured by quantitative PCR and relative gene expression of equine MMP-1, MMP-3, MMP-13 and Aggrecanase (AGGRase). Expression of all genes was compared to the expression levels of equine GAPDH. RNA was extracted from cells on day 7 using the QIAshredder and RNeasy mini kit (Qiagen, Valencia, Calif.). Complimentary DNA was generated using 50 ng of total RNA and oligo(dT) primers with the SuperScript III first-strand synthesis kit (Invitrogen, Carlsbad, Calif.). Equine MMP-1, MMP-3, MMP-13, and GAPDH primer/probes were obtained from the Lucy Whittier Molecular Core Facility (UC Davis, Davis, Calif.). Sequences for these genes are as follows:

MMP-1 forward 5′-AAGCTGCTTATGAGGTTTCCCA-3′ (SEQ ID NO:4),
reverse 5′-GGGTATCCGTAGAGCACATCCT-3′ (SEQ ID NO:5),
probe 5′-FAM-AGCCCAGTACTTATTACCTTTGAAAAACCGGAC-TAMRA-3′ (SEQ ID NO:6);
MMP-3 forward 5′-AACACTGGACGAAGGATGCAT-3′ (SEQ ID NO:7),
reverse 5′-ACCCAGGGAATGACCAAGTTC-3′ (SEQ ID NO:8),
probe 5′-FAM-AGGGATCAATTTCTCCTTGTTGCTGCTCA-TAMRA-3′ (SEQ ID NO:9). Aggrecanase sequences were:
forward 5′-GCCTTCACT GCTGCTCATGA-3′ (SEQ ID NO:1),
reverse 5′-CCAACACATGGCTTTGAATTGT-3′ (SEQ ID NO:2),
and probe 5′-FAM-CTGGGCCATGTCTTCAACATGCTCC-TAMRA-3′ (SEQ ID NO:3). Quantitative PCR was run using 2.5 ng RNA per sample (in duplicate) on a 384-well Roche Light Cylcer 480 (Roche, Indianapolis, Ind.) with a 95° C., 3 minute hold; followed by 40 cycles of a 95° C., 10 second denaturation, a 60° C., 30 second anneal, and a 72° C., 1 second extension with a single acquisition mode. Sample cycle threshold data were normalized to standards for each gene and compared to each other with a gene of interest to GAPDH ratio.

### Results

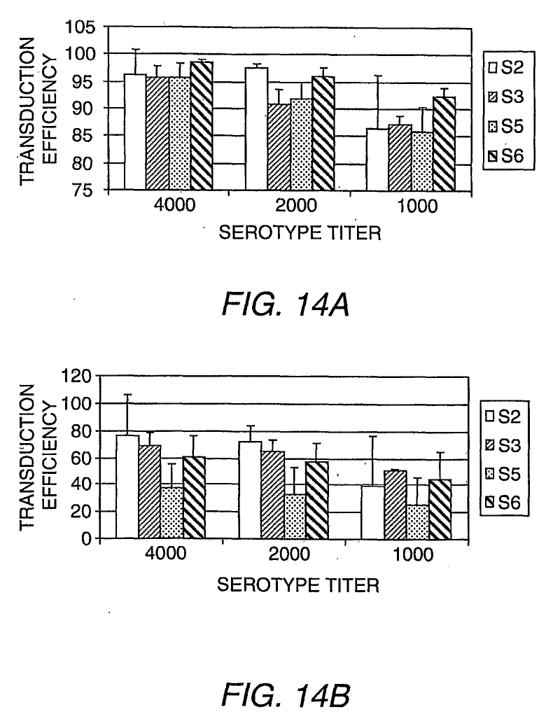

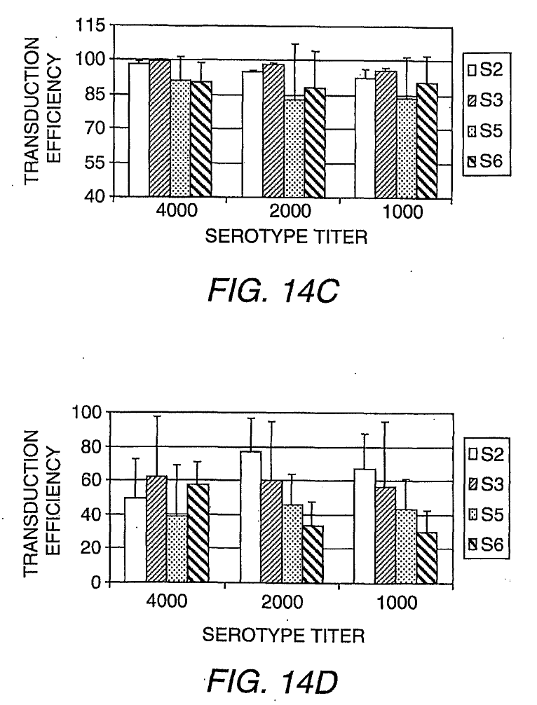

The transduction efficiency at days 7 and 14 of chondrocytes by various serotypes of AAV-GFP are depicted in FIGS. 14A and 14B respectively. The transduction efficiency at days 7 and 14 of synoviocytes by various serotypes of AAV-GFP are depicted in FIGS. 14C and 14D respectively.

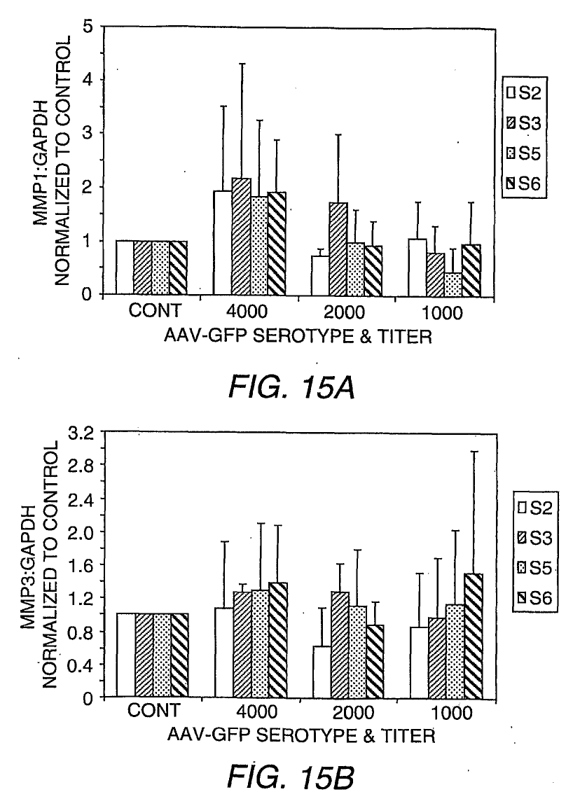

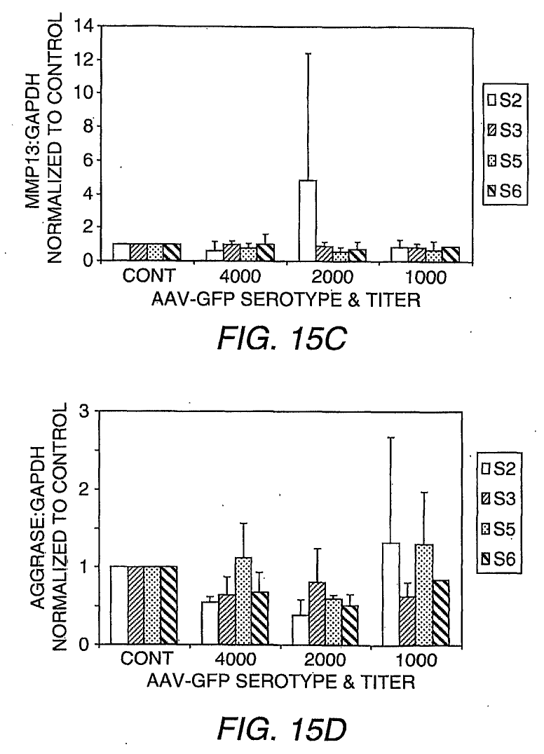

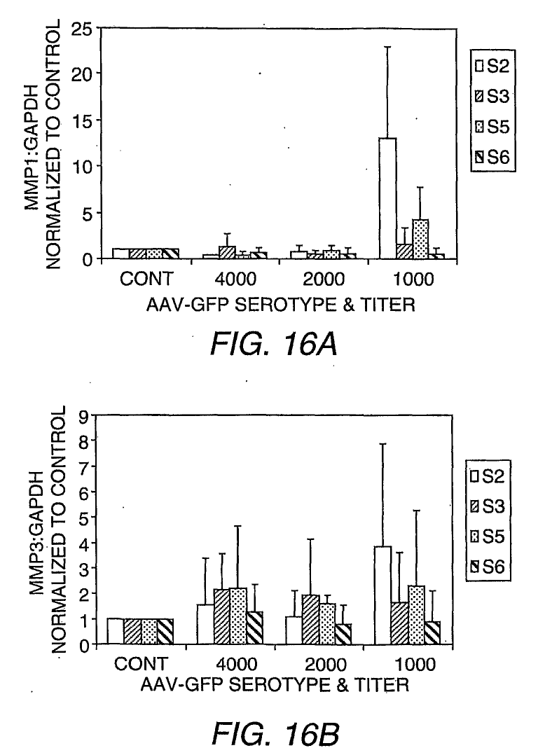

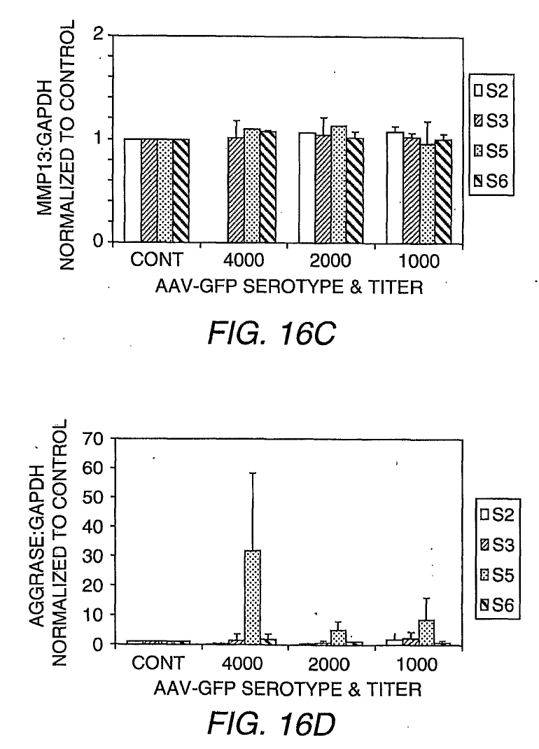

The inflammatory profiles, as measured by RNA expression of the inflammatory molecules MMP1, MMP3, MMP13 and AGGRase, in chondrocytes of AAV-GFP serotypes 2, 3, 5 and 6, are shown in FIGS. 15A-D, respectively. The inflammatory profiles, as measured by RNA expression of the inflammatory molecules MMP1, MMP3, MMP13 and AGGRase, in synoviocytes of AAV-GFP serotypes 2, 3, 5 and 6, are shown in FIGS. 16A-D, respectively.

### Harvest and Culture of Equine Bone Marrow Stem Cells

Bone marrow mesenchymal stem cells (MSCs) were collected from both the sternum and iliac crest of 2-5 year old equine subjects. A solution of 5000× heparin was made by filling a 60 ml sterile syringe with 3 ml of 10000× heparin and 3 ml of sterile filtered 0.9% saline solution. Nucleated cells were removed by centrifuging samples at 100 g for 1 min. The top layer (serum) was removed and placed into a new 15 ml conical centrifuge tube. The marrow serum was spun at 200 g for 5 min to pellet nucleated cells. The serum was removed from the cell pellet and added back to the original marrow-blood tube. The cell pellet was resuspended in 1×PBS, filtered through a 70 μm cell strainer, and a 1:10 dilution of cells in NH₄Cl was counted for determining nucleated cell populations. The PBS cell solution was then saved for later cell seeding. The original marrow-blood tube was mixed and subjected to the above process two more times to generate two more aliquots of nucleated cells in suspension with PBS. The aliquots were centrifuged at 1000 g for 10 min to collect all nucleated cells and red blood cells in the suspension. After centrifugation, the cell pellets were combined and resuspended with seeding medium (DMEM (GIBCO, Grand Island, N.Y.), 10% FBS (HyClone, Logan, Utah), 25 mM HEPES (GIBCO, Grand Island, N.Y.), 300 μg/ml L-glutamine, 1 mM Na pyruvate (GIBCO, Grand Island, N.Y.), and 50 U/ml penicillin/50 μg/ml streptomycin (GIBCO, Grand Island, N.Y.)) into culture flasks in the following cell densities: T25 at 7.0×10⁶ cells, T75 at 20×10⁶ cells, and T150 at 40×10⁶ cells. Cells were grown at 37° C. with 5% CO₂ until colonies formed.

Bone marrow stem cell colonies were lifted from growth flasks by first washing cells with PBS and trypsinizing cells for 90 seconds. Cells were suspended with seeding medium, counted with a hemacytometer, and centrifuged at 200 g for 5 min to pellet bone marrow stem cells. The pellet was then resuspended in expansion medium (α-MEM (GIBCO, Grand Island, N.Y.), 10% FBS, 25 mM HEPES, 300 μg/ml L-glutamine, 1 mM Na pyruvate, and 50 U/ml penicillin/50 μg/ml streptomycin) and seeded into culture flasks at 25-30% confluency. Similar to the following: T25 at 480,000 cells and T75 at 1.5×10⁶ cells. Cells were allowed to expand for 24-36 hours, harvested by trypsinizing, and reseeded into larger culture flasks with expansion medium. Cells were passaged no more than three times from out of colonies before cells were harvested and frozen in freeze medium (A-MEM with 85% FBS and 5% DMSO (Sigma, St. Louis, Mo.)) and stored in liquid nitrogen until used for assays. For assays, cells were removed from liquid nitrogen storage and quickly thawed in a 37° C. water bath. Cells were suspended in seeding medium, counted, and centrifuged at 200 g for 5 min. Cell pellets were resuspended in expansion medium to generate monolayer cultures at 30% confluency (30%=15,000 cells/cm²) and plated in 48-well plates (Corning Inc, Corning, N.Y.).

### Screening of AAV Serotypes (S1-S8)

MSCs were seeded in duplicate wells of a 48-well culture plate and transduced with 4000 particles/cell of each scAAV-GFP serotype (S1-S8). Briefly, cells were washed with 0.5 ml 1×PBS and virus was added as above in 0.2 ml DMEM. Cells were allowed to uptake virus for 4 hours at 37° C. in the serum-free DMEM and then nourished with seeding medium to a final volume of 0.5 ml. Growth medium was replaced every 48 hr in 0.5 ml volumes. Fluorescence was measured daily using a SPECTRAmax GEMINI-XS fluorometer with excitation at 472 nm, emission 512 nm, and a 495 nm emission cutoff filter. The entire well surface was scanned using 10 reads/well. Fluorometer readings were discontinued at day 20 due to inconsistent readings. GFP fluorescence was also monitored using an Olympus IX70 fluorescent microscope with a 20× optical plus a 1.5× extra zoom and a WU filter that has an excitation from 330-385 nm and an emission >420 nm. Images were obtained with this scope and BioQuant software every 3 days for the first 20 days. Cells were assessed for transduction efficiency and viability when ˜100% confluent on days 3, 7, 10, 14, 17, and 20 days by treatment with 0.25% trypsin (Invitrogen, Carlsbad, Calif.) and counting fluorescing cells on a hemacytometer with the fluorescence microscope and Trypan Blue staining.

### Results

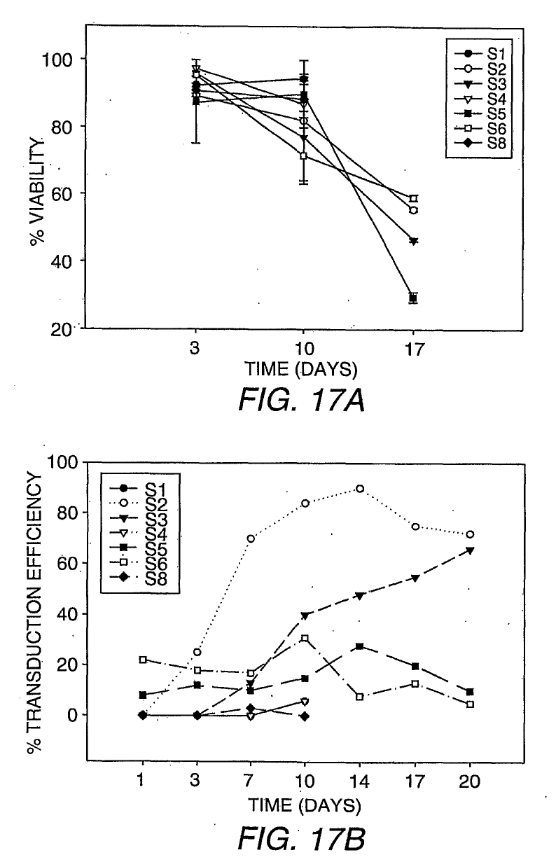

Transduction images of AAV-GFP-transduced MSCs at 4000 particles/cell were compiled at day 10 post-transduction. S2 produced the best fluorescence with S3 at approximately 50% that of S2 and S5 and S6 at 20% of S2. S3 transduction efficiency appears to increase over time (FIG. 17B). Viability of the AAV-GFP-transduced MSCs was between 60-100% until day 17 when the MSCs appeared overgrown and started lifting from the culture plates (FIG. 17A). No values are shown for AAV-GFP serotypes S1, S4, and S8 past day 10, apparently due to little or no transduction of the MSCs.

The foregoing is illustrative of the present invention, and is not to be construed as limiting thereof. The invention is defined by the following claims, with equivalents of the claims to be included therein

TABLE 1

| | GenBank Accession |
|---|---|
| | Number |
| Complete Genomes | |
| Adeno-associated virus 1 | NC_002077, AF063497 |
| Adeno-associsted virus 2 | NC_001401 |
| Adeno-associated virus 3 | NC_001729 |
| Adeno-associated virus 3B | NC_001863 |
| Adeno-associated virus 4 | NC_001829 |
| Adeno-associated virus 5 | Y18065, AF085716 |
| Adeno-associated virus 6 | NC_001862 |
| Avian AAV ATCC VR-865 | AY186198, AY629583, |
| | NC_004828 |
| Avian AAV strain DA-1 | NC_006263, AY629583 |
| Bovine AAV | NC_005889, AY388617 |
| Clade A | |
| AAVI | NC_002077, AF063497 |
| AAV6 | NC_001862 |
| Hu.48 | AY530611 |
| Hu43 | AY530606 |
| Hu44 | AY530607 |
| Hu46 | AY530609 |
| Clade B | |
| Hu.19 | AY530584 |
| Hu.20 | AY530586 |
| Hu23 | AY530589 |
| Hu22 | AY530588 |
| Hu24 | AY530590 |
| Hu21 | AY530587 |
| Hu27 | AY530592 |
| Hu28 | AY530593 |
| Hu29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Flu T88 | AY695375 |
| Hu T71 | AY695374 |
| Hu T70 | AY695373 |
| Hu T40 | AY695372 |
| Flu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| P11 | AY530553 |
| P13 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 (AAV9) | AY530579 |
| Hu31 | AY530596 |
| Hu32 | AY530597 |
| Clonal Isolate | |
| AAV5 | Y18065, AF085716 |
| AAV3 | NC_001729 |
| AAV3B | NC_001863 |
| AAV4 | NC_001829 |
| Rh34 | AY243001 |
| Rh33 | AY243002 |
| Rh32 | AY243003 |

We claim:

1. A method of delivering a nucleic acid to a connective tissue cell, the method comprising contacting the cell with a virus vector comprising:
(a) an adeno-associated virus (AAV) capsid; and
(b) a recombinant nucleic acid comprising 5′ and 3′ AAV terminal repeats and a heterologous nucleotide sequence, wherein the recombinant nucleic acid is packaged within the AAV capsid.

2. The method of claim 1, wherein the cell is a joint tissue cell or a precursor thereof.

3. The method of claim 1, wherein the cell is a synoviocyte, chondrocyte or a fibrocartilage cell.

4. The method of claim 1, wherein the cell is a bone marrow derived mesenchymal stem cell.

5. The method of claim 1, wherein the cell is contacted with the virus vector in vitro.

6. The method of claim 1, wherein the heterologous nucleotide sequence encodes a polypeptide.

7. The method of claim 6, wherein the polypeptide is a therapeutic polypeptide.

8. The method of claim 7, wherein the therapeutic polypeptide is a growth factor, an anti-catabolic factor, or a combination thereof.

9. The method of claim 7, wherein the therapeutic polypeptide is an insulin-like growth factor I and/or II, an interleukin receptor antagonist protein (IRAP), a transforming growth factor β, a bone morphogenic protein, VEGF and/or RANKL, or any combination thereof.

10. The method of claim 1, wherein the AAV capsid is an AAV1, AAV2, AAV3, AAV4, AAV5, AAV6, AAV7, AAV8, AAV9, AAV10, AAV11 or AAV12 capsid.

11. The method of claim 1, wherein the AAV terminal repeats are AAV1, AAV2, AAV3, AAV4, AAV5, AAV6, AAV7, AAV8, AAV9, AAV10, AAV11 or AAV12 terminal repeats.

12. The method of claim 1, wherein the AAV capsid is an AAV2, AAV3 or AAV6 capsid and, optionally, the cell is a chondrocyte and/or a synoviocyte.

13. The method of claim 1, wherein the virus vector is a duplexed parvovirus vector, wherein the recombinant nucleic acid comprises the AAV terminal repeats, the heterologous nucleotide sequence, and a non-resolvable terminal repeat.

14. A method of delivering a nucleic acid to a connective tissue of a subject, the method comprising administering to the subject a cell produced according to the method of claim 5.

15. The method of claim 14, wherein a pharmaceutical composition comprising the virus vector and a pharmaceutically acceptable carrier is administered to the subject.

16-24. (canceled)

25. The method of claim 14, wherein the method comprises:
(a) removing a cell from the subject;
(b) introducing the virus vector into the cell and/or a progeny thereof; and
(c) administering the cell of (b) and/or a progeny thereof to the subject.

26-28. (canceled)

29. A method of treating a connective tissue disorder in a subject, the method comprising administering to a subject in need thereof an effective amount of a cell produced according to the method of claim 5.

30-44. (canceled)

45. The method of claim 29, wherein the method comprises:
(a) removing a cell from the subject;
(b) introducing the virus vector into the cell and/or a progeny thereof; and
(c) administering the cell of (b) and/or a progeny thereof to the subject.

46-49. (canceled)

50. A method of administering a nucleic acid to a connective tissue in a subject, the method comprising administering to the subject a virus vector comprising:
(a) an adeno-associated virus (AAV) capsid; and
(b) a recombinant nucleic acid comprising 5′ and 3′ AAV terminal repeats and a heterologous nucleotide sequence, wherein the recombinant nucleic acid is packaged within the AAV capsid.

51-71. (canceled)

72. A method of treating a connective tissue disorder, the method comprising administering to a subject in need thereof an effective amount of a virus vector comprising:
(a) an adeno-associated virus (AAV) capsid; and
(b) a recombinant nucleic acid comprising 5′ and 3′ AAV terminal repeats and a heterologous nucleotide sequence, wherein the recombinant nucleic acid is packaged within the AAV capsid.

73-90. (canceled)
